(19) United States
(12) Patent Application Publication
(10) Pub. No.: US 2025/0262050 A1
(43) Pub. Date: Aug. 21, 2025

(54) Valve Retention Features

(71) Applicant: St. Jude Medical, Cardiology Division, Inc., St. Paul, MN (US)

(72) Inventors: Tyler Govek, St. Louis Park, MN (US); Neil Theisen, Champlin, MN (US); William H. Peckels, Robbinsdale, MN (US); Jay Reimer, Shoreview, MN (US); Victoria Schuman, Minneapolis, MN (US); Jeffrey Paul LaPlante, Minneapolis, MN (US); Brandon Dennis, St. Paul, MN (US)

(73) Assignee: St. Jude Medical, Cardiology Division, Inc., St. Paul, MN (US)

(21) Appl. No.: 18/955,714

(22) Filed: Nov. 21, 2024

Related U.S. Application Data
(60) Provisional application No. 63/554,326, filed on Feb. 16, 2024.

(51) Int. Cl. A61F 2/24

(52) U.S. Cl. CPC A61F 2/2433

(57) ABSTRACT

A system for delivering a prosthetic heart valve may include, a handle, an outer catheter, an inner catheter, a nose cone coupled to the inner catheter, and an inflatable balloon having a proximal leg coupled to the outer catheter and a distal leg coupled to the nose cone or the inner catheter. A first valve retainer may be coupled to the inner catheter and positioned within the balloon. In an assembled state of the first valve retainer, an inner stop is directly fixed to the inner catheter and an outer stop is positioned around and coupled to the inner stop. The inner stop has a maximum outer diameter that is smaller than a maximum inner diameter of the distal leg. In the assembled condition of the first valve retainer, the outer stop has a maximum outer diameter that is larger than the maximum inner diameter of the distal leg.

### CROSS-REFERENCE TO RELATED APPLICATIONS

This application claims priority to the filing date of U.S. Provisional Patent Application No. 63/554,326, filed Feb. 16, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

Generally, this disclosure relates to features for use in balloon catheters for use in delivering and/or deploying balloon-expandable prosthetic heart valves. In some examples, a balloon-expandable prosthetic heart valve is crimped over a balloon of a balloon catheter prior to being inserted into the patient's vasculature where it is advanced through the vasculature to the native heart valve being treated. At various points during the procedure, including while passing the prosthetic heart valve through an introducer leading to the patient's vasculature, as well as advancement through tortuous vasculature such as an approximately 180-degree bend of the aortic arch, there is an increased chance that the prosthetic heart valve is shifted or otherwise dislocated from its position relative to the balloon of the balloon catheter. In some examples, to help reduce the likelihood of such shifting or dislocation, components may be provided internal to the balloon or on the balloon to help maintain the position of the prosthetic heart valve relative to the balloon during the delivery. In some examples in which components are provided internal to the balloon, sizing of the balloon and limits on the ability to stretch the balloon result in difficulties in inserting valve retainer features into the balloon while allowing the valve retainer features to have a large enough size to assist with maintaining the prosthetic heart valve in the desired position. Various example provided herein allow for valve retainer(s) to be inserted into the balloon while having a relatively small profile, and then expanding or otherwise changing configuration to a larger profile to better function to retain the prosthetic heart valve in the desired positioning on the balloon during delivery.

According to one example of the disclosure, a system for delivering a prosthetic heart valve includes a handle, an outer catheter extending from the handle, an inner catheter extending from the handle and through an interior of the outer catheter, a nose cone coupled to a distal end of the inner catheter, and an inflatable balloon having a proximal leg coupled to the outer catheter and a distal leg coupled to (i) the nose cone or (ii) the inner catheter. A first valve retainer may be coupled to the inner catheter and positioned within the balloon. The first valve retainer may have an assembled state in which an inner stop is directly fixed to the inner catheter and an outer stop is positioned around and coupled to the inner stop. The inner stop may have a maximum outer diameter that is smaller than a maximum inner diameter of the distal leg, and in the assembled condition of the first valve retainer, the outer stop may have a maximum outer diameter that is larger than the maximum inner diameter of the distal leg. The inner stop may include a ramped outer surface portion that increases in diameter in a proximal-to-distal direction of the inner stop. The outer stop may include a rim and a plurality of extensions extending from the rim. The outer catheter may be directly coupled to the rim. When the first valve retainer is in an unassembled condition, and in the absence of applied forces, the outer stop may have a maximum outer diameter that is smaller than the maximum inner diameter of the distal leg. The inner stop may include a shoulder at a distal end of the ramped outer surface portion. At least one of the plurality of extensions may include an interiorly extending prong at a distal end thereof, the interiorly extending prong configured to engage the shoulder in the assembled condition of the first valve retainer. In the assembled condition of the first valve retainer, each of the plurality of extensions may flare radially outwardly relative to the rim. Each of the plurality of extensions may be coupled to the rim via a living hinge. The balloon may include a proximal pillow, a distal pillow, and a central portion between the proximal pillow and the distal pillow. The proximal pillow and the distal pillow may each have a diameter that is greater than a diameter of the central portion, the central portion being configured to receive the prosthetic heart valve in a crimped condition. The first valve retainer may be positioned inside the proximal pillow. A distal end of the outer catheter may extend into the proximal pillow. The outer catheter may include a catheter wall, and a plurality of apertures may be formed within the catheter wall, the plurality of apertures being positioned within the proximal pillow.

According to another example of the disclosure, a method of manufacturing a delivery catheter may include providing a balloon having a distal leg and a proximal leg, and inserting an inner catheter into an interior of the balloon so that an inner stop fixed to the inner catheter passes through the distal leg, the inner stop having a maximum outer diameter that is smaller than a maximum inner diameter of the distal leg. The method may also include inserting an outer stop into the interior of the balloon so that the outer stop passes through the distal leg while the inner catheter is received through the outer stop. During inserting the outer stop, the outer stop may have a maximum outer diameter that is smaller than the maximum inner diameter of the distal leg. While the inner stop and the outer stop are both within the interior of the balloon, the outer stop may be advanced over the inner stop to increase the maximum outer diameter of the outer stop, the increased maximum outer diameter of the stop being larger than the maximum inner diameter of the distal leg. The inner stop may have a ramped outer surface portion and the outer stop may have a rim and a plurality of extensions extending from the rim, and advancing the outer stop over the inner stop may include advancing the outer stop over the ramped outer surface portion to force the plurality of extensions to flare outwardly relative to the rim. The inner stop may include a shoulder, and at least one of the plurality of extensions may include an interiorly extending prong. In the absence of applied forces, the outer stop may have the maximum outer diameter that is smaller than the maximum inner diameter of the distal leg. The method may also include continuing to advance the outer stop over the inner stop until the at least one interiorly extending prong advances past the shoulder. When the at least one interiorly extending prong advances past the shoulder, the corresponding extension may snap inwardly toward the inner catheter such that the at least one interiorly extending prong engages the shoulder to prevent the outer stop from retracting relative to the inner stop. The method may also include advancing an outer catheter through the proximal leg into the interior of the balloon. The rim may be fixed to a distal end of the outer catheter, and inserting the outer stop into the interior of the balloon may be performed by advancing the outer catheter toward the balloon.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of a handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is a side view of an example of the balloon of FIG. 6 isolated from other components of the delivery system.

FIG. 13A is a side view of an example of a distal end of the delivery catheter of FIG. 4 in a state of assembly.

FIGS. 13B-13C are side and front views, respectively, of an example of a proximal inner stop of FIG. 13A.

FIGS. 13D-13E are side and rear views, respectively, of an example of a distal stop of FIG. 13A.

FIG. 13F is a side view of an example of a distal end of the delivery catheter of FIG. 4 in a state of assembly with the balloon of FIG. 12, the proximal inner stop of FIGS. 13B-13C, and the distal stop of FIGS. 13D-13E.

FIGS. 13G-13H are side and front views, respectively, of an example of a proximal outer stop.

FIGS. 13I-13J are side and front views, respectively, of the proximal outer stop of FIGS. 13G-13H assembled to the proximal inner stop of FIGS. 13B-13C.

FIG. 13K is a side view of an example of a distal end of the delivery catheter of FIG. 4 in a state of assembly with the proximal outer stop of FIGS. 13G-13H assembled to the proximal inner stop of FIGS. 13B-13C.

FIG. 13L is a flow chart showing an example of a method of assembling a distal end of a delivery catheter.

FIG. 14 is a highly schematic view of another example of an assembled proximal stop.

FIG. 15A is a highly schematic perspective view of another example of an assembled proximal stop.

FIGS. 15B-15C are highly schematic cross-sections of a proximal outer stop of the assembled proximal stop of FIG. 15A in collapsed and expanded configurations, respectively.

FIG. 16 is a side view of an example of a distal end of the delivery catheter of FIG. 4 with a pusher feature engaged with a proximal stop.

FIG. 17 is a side view of an example of a distal end of the delivery catheter of FIG. 4.

FIG. 18A is a side view of an example of an inner stop.

FIG. 18B is a front view of an example of an outer stop for use with the inner stop of FIG. 18A.

FIG. 18C is a schematic view of an example step of assembling the outer stop of FIG. 18B to the inner stop of FIG. 18A.

FIG. 18D is a cross-section of the outer stop of FIG. 18B assembled to the inner stop of FIG. 18A.

FIG. 19A is a side view of an example of an inner stop.

FIG. 19B is a front view of an example of an outer stop for use with the inner stop of FIG. 19A.

FIG. 19C is a schematic view of an example step of assembling the outer stop of FIG. 19B to the inner stop of FIG. 19A.

FIG. 19D is a cross-section of the outer stop of FIG. 19B assembled to the inner stop of FIG. 19A.

FIG. 20A is a highly schematic illustration of an example of a stop in a relaxed condition.

FIG. 20B is a highly schematic illustration of the stop of FIG. 20A in a tensioned configuration.

FIG. 21A is a highly schematic cross-section of an example of a stop in an uncompressed condition.

FIG. 21B is a highly schematic cross-section of the stop of FIG. 21B in a compressed condition.

FIG. 22A is a highly schematic cross-section of an example of an inner stop and outer stop in a state of assembly.

FIG. 22B is a highly schematic cross-section of the outer stop and inner stop of FIG. 22A assembled together with the outer stop in an expanded condition.

FIG. 23A is a highly schematic view of an example of a stop in an uncompressed condition.

FIG. 23B is a highly schematic view of the stop of FIG. 23B in a compressed condition.

FIG. 24 is a highly schematic view of an example of a stop in an uncompressed condition.

FIG. 25 is a highly schematic cross-section of an example of a distal end of the delivery catheter of FIG. 4.

FIG. 26A is a highly schematic side view of an example of the distal end of the delivery catheter of FIG. 4 with a first friction-enhancing surface.

FIG. 26B is a highly schematic side view of an example of the distal end of the delivery catheter of FIG. 4 with a second friction-enhancing surface.

FIG. 27 is a highly schematic side view of an example of a balloon of the delivery catheter of FIG. 4 with a friction-enhancing features.

FIG. 28 is a highly schematic side view of an example of a balloon of the delivery catheter of FIG. 4 with studs or nodules in contact with a prosthetic heart valve crimped on the balloon.

FIG. 29 is a highly schematic side view of an example of a balloon of the delivery catheter of FIG. 4 with microbeads received therein.

FIG. 30 is a highly schematic cut-away view of an example of a distal end of the delivery catheter of FIG. 4 with non-expandable stops within the balloon.

FIG. 31A is a highly schematic perspective view of a distal end of an example of a spreadable outer balloon catheter.

FIG. 31B is a highly schematic cross-section of an example of the distal end of the delivery catheter of FIG. 4 incorporating the spreadable outer balloon catheter of FIG. 31A.

FIG. 32 is a highly schematic cross-section of an example of a balloon with overhangs.

FIG. 33 is a highly schematic cross-section of an example of a balloon with retainer features mounted on the balloon.

FIGS. 34A-34C are highly schematic cross-sections of an example method of forming an example of a valve retainer within a balloon.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

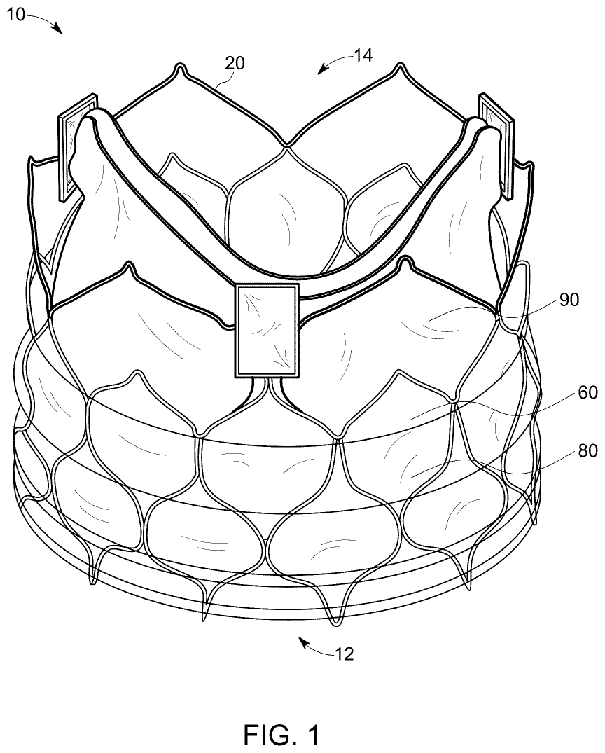

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

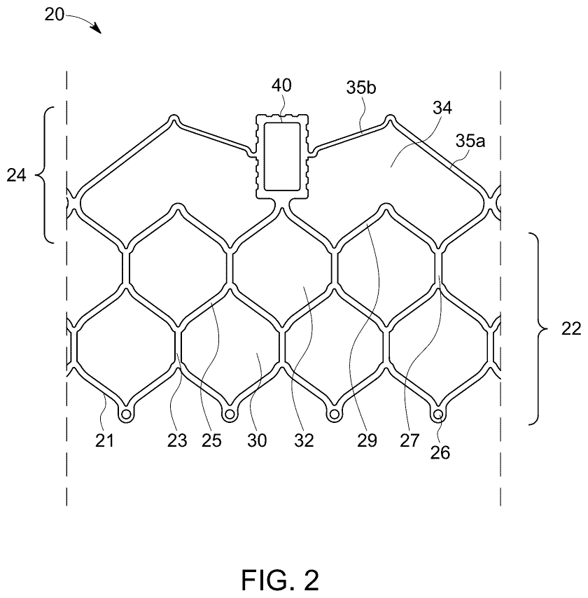

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with scaling, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

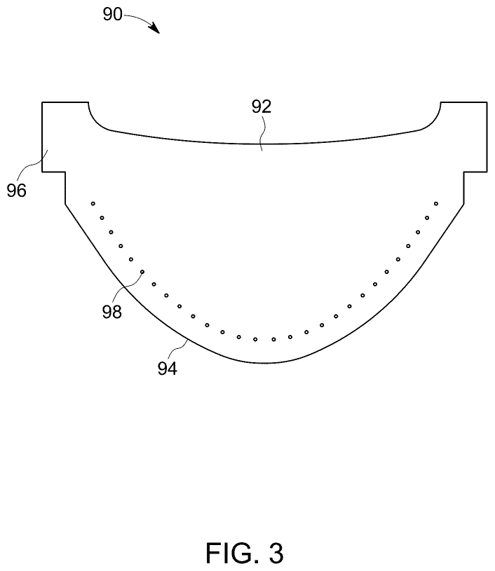

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g., via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Leaflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

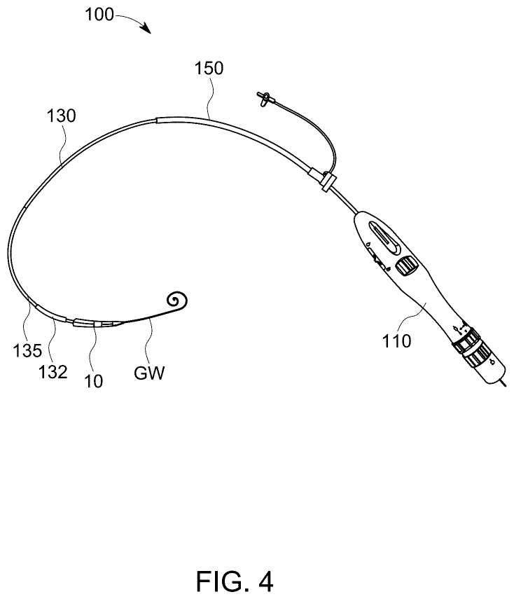

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 (which may also be referred to as a “nose cone”) of the delivery catheter 130. The guidewire GW may be introduced into the patient to the desired location, and the delivery system 100 may be introduced over the guidewire GW to help guide the delivery catheter 130 through the patient's vasculature over the guidewire GW.

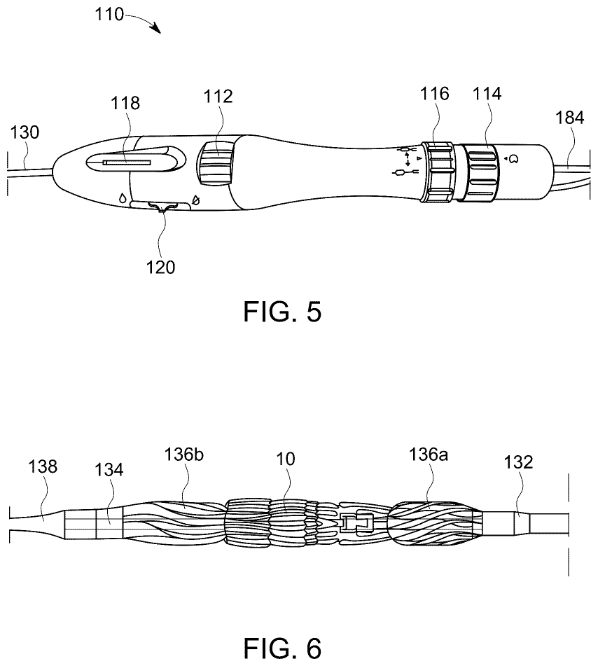

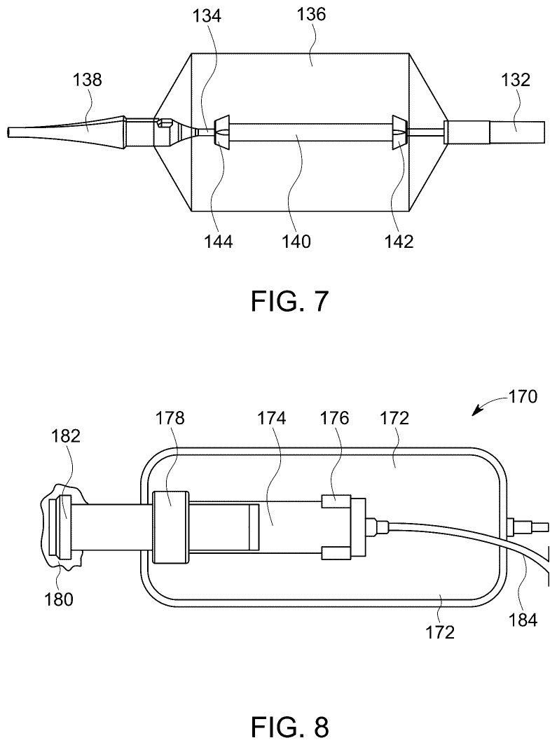

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

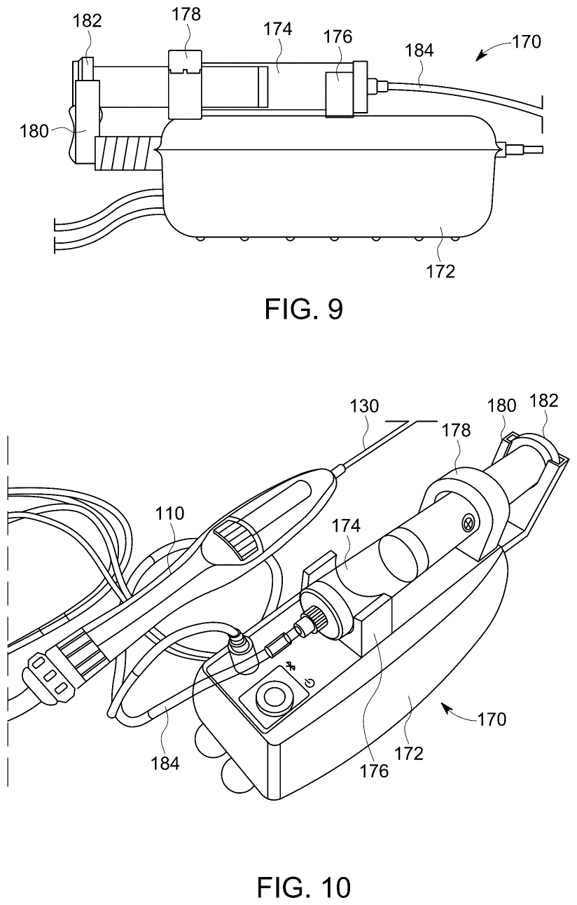

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. As used herein, the phrase “fluid reservoir” and “syringe” may be used interchangeably. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

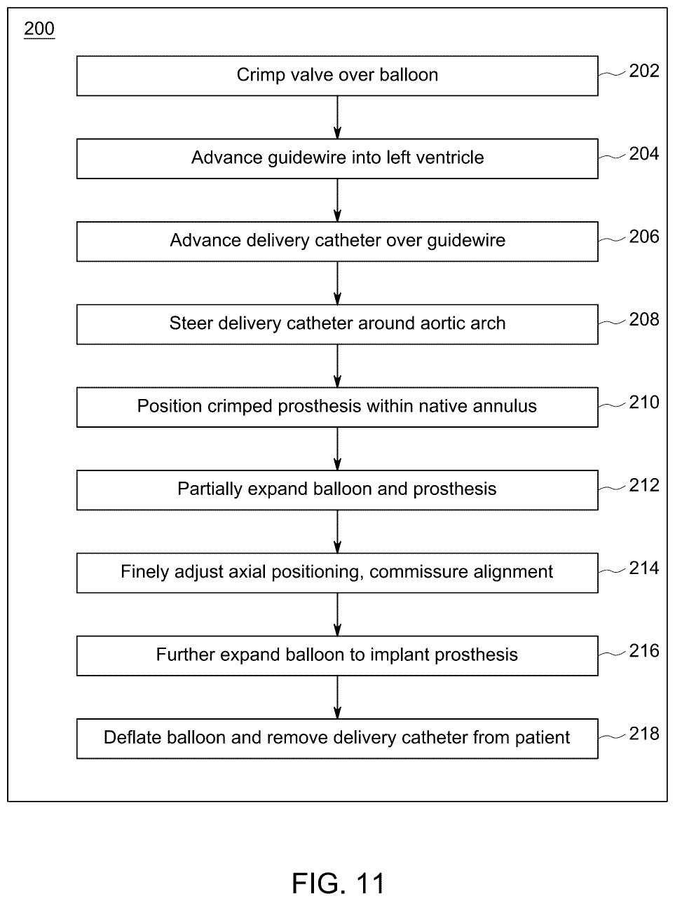

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieve and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

As noted above, for example in connection with FIG. 7, in some examples it may be desirable to include one or more features within the balloon 136, including on the inner catheter 134, to contact opposite ends of the prosthetic heart valve 10 when the prosthetic heart valve 10 is crimped over the balloon 136. Such features, including for examples stops 142, 144, may help maintain the prosthetic heart valve 10 retain its axial position on the balloon 136 as the prosthetic heart valve 10 is passed through the introducer 150 and/or through the patient's vasculature. In some examples, stops 142, 144 that have relatively large radial extents (or diameters) may provide better valve retention than stops 142, 144 that have relatively small radial extents (or diameters). However, one potential limitation on providing a stop with a relatively large radial extent is that, during manufacturing, the balloon 136 is typically pulled over the inner catheter 134 so that the ends of the balloon 136 may be fixed to the delivery device (e.g., a proximal end of the balloon 136 may be fixed to outer catheter 132 and a distal end of the balloon 136 may be fixed to nose cone 138). In some examples, this limitation exists because the balloon 136 is formed separately from other components of the delivery system 100.

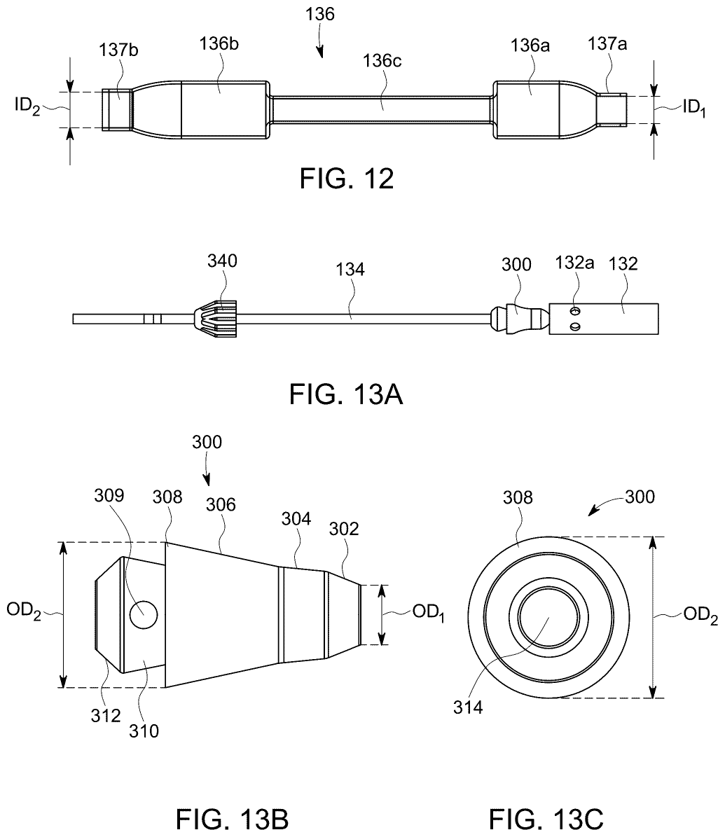

Referring now in addition to FIG. 12, FIG. 12 illustrates an example of a balloon 136. Balloon 136 is shown in this example in a formed state but prior to coupling the balloon 136 to another component of delivery system 100. As noted in connection with FIG. 6, in some examples balloon 136 may be formed and processed to include a proximal pillow 136a and a distal pillow 136b that may abut the opposite axial ends of the prosthetic heart valve 10 when the prosthetic heart valve 10 is crimped over the crimp zone 136c of the balloon 136. An example of possible contact between the opposite ends of the prosthetic heart valve 10 and the proximal pillow 136a and distal pillow 136b is shown in FIG. 6. Although the proximal pillow 136a and distal pillow 136b may in some examples help the prosthetic heart valve 10 retain its axial position on the balloon 136 during delivery (including while passing the prosthetic heart valve through introducer 150), it may be desirable in some examples to provide one or more additional valve retention features. However, if such one or more additional valve retention features is to be located within the interior of the balloon 136, it may in some examples be helpful for the outer diameter of such features to be smaller than the inner diameter ID₁ of the proximal balloon leg 137a and/or smaller than the inner diameter ID₂ of the distal balloon leg 137b. For example, the proximal balloon leg 137a and the distal balloon leg 137b may be unable to stretch to any meaningful degree to allow them to slide over a component that has an outer diameter that is larger than the inner diameter of the respective balloon leg.

In some examples, the above restriction may be overcome by providing a stop (the term “stop” may be used interchangeably herein with the phrase “valve retainer”) that is sized for insertion through the opening defined by proximal leg 137a (or distal leg 137b), but which can expand in diameter once positioned within the balloon 136.

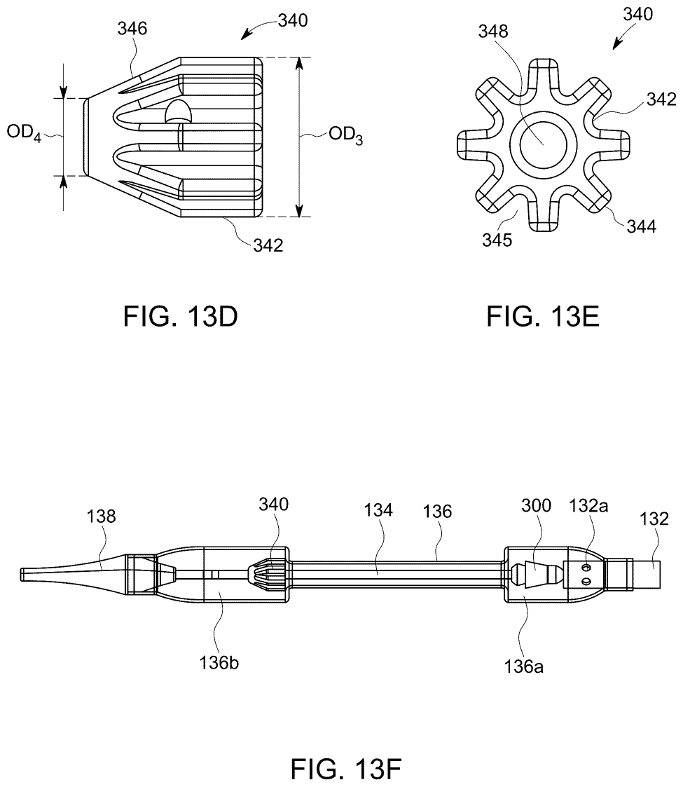

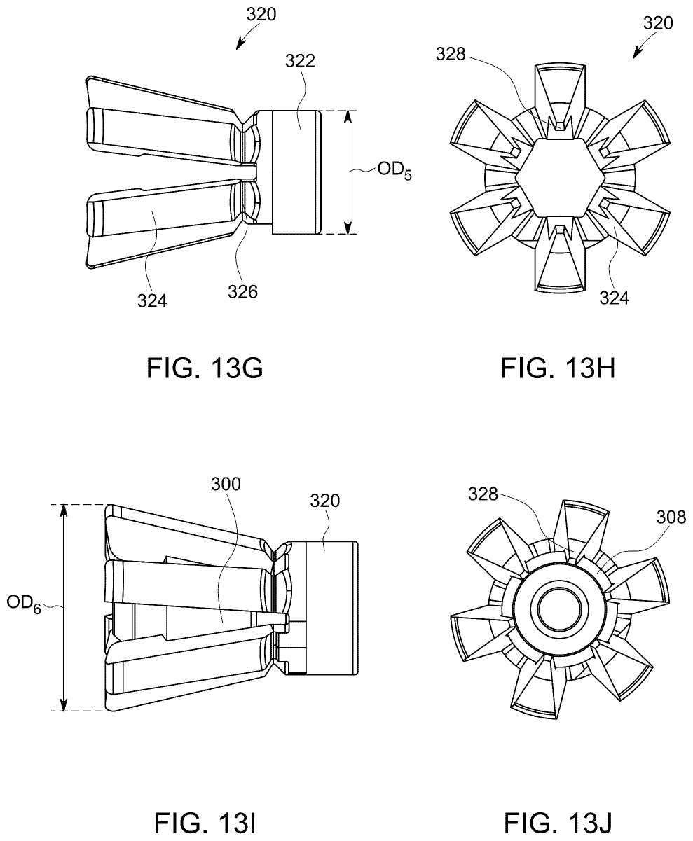

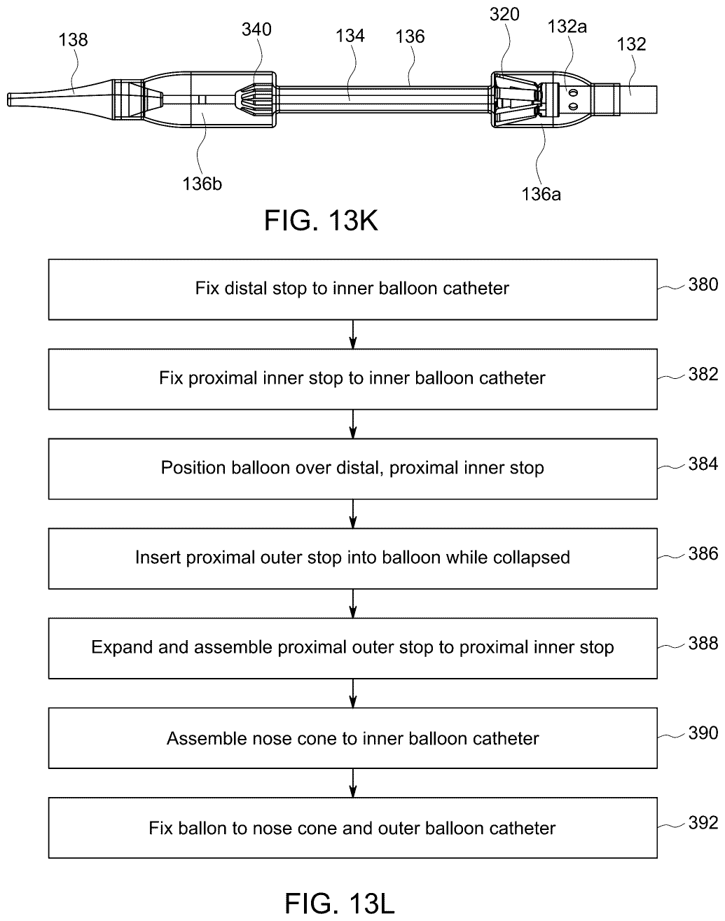

Referring now to FIG. 13A, FIG. 13A is an example of a distal end portion of delivery catheter 130, prior to the delivery catheter 130 being fully assembled. In particular, in the example of FIG. 13A, the valve retainers (described in further detail below) have not been fully assembled, the nose cone 138 has not yet been coupled to the inner balloon catheter 134, and the balloon 136 has not been coupled to any portion of the delivery catheter 130. In the illustrated example, the outer balloon catheter 132 may have one or more apertures 132a formed in a wall thereof near a distalmost end of the outer balloon catheter 132, the one or more apertures 132a configured to be positioned at or near a proximal end of proximal pillow 136a when the device is assembled. As is described in greater detail below, such apertures 132a in some examples may assist with enhanced de-airing of the balloon 136.

In the illustration of the example of the stage of assembly shown in FIG. 13A, an inner proximal stop 300 and a distal stop 340 are already coupled to inner balloon catheter 134. In some examples, inner proximal stop 300 and distal stop 340 may be formed integrally with the inner balloon catheter 134, such as by injection molding inner balloon catheter 134 together with inner proximal stop 300 and distal stop 340. In some examples, inner proximal stop 300 and distal stop 340 may be formed in a second step after forming the inner balloon catheter 134, such as by overmolding the inner proximal stop 300 and the distal stop 340 onto the inner balloon catheter 134. In some examples, inner proximal stop 300 and distal stop 340 may be fixed to the inner balloon catheter 134, for example via adhesives.

Now referring in addition to FIGS. 13B-13C, FIGS. 13B-13C show side and front views, respectively, of an example of inner proximal stop 300 in isolation. In the illustrated example, proximal inner stop 300 may include a plurality of sections, such as five sections. A proximalmost first section 302 in some examples has a proximal end defining an outer diameter OD₁, which tapers outwardly in a distal direction to a second section 304, which tapers outwardly in the distal direction to a third section 306, which ends in a distalmost section having an outer diameter OD₂, which is larger than outer diameter OD₁. In the illustrated example, the first section 302 and third section 306 may have tapers that slope at larger angles compared to second section 304, although in other examples other relative levels of slope may be provided. In some examples, one or more of the first section 302, second section 304, and third section 306 may be combined to provide fewer than three distinct sections. The third section 306 in some examples terminates in a shoulder 308 that abruptly decreases in outer diameter into a fourth section 310, which may taper outwardly in the distal direction to a fifth section 312, which may taper back inwardly to a distalmost end of the inner proximal stop. In some examples, a hole, such as port hole 309, may be provided on the inner proximal stop 300, for example in the fourth section 310, to allow for glue or other adhesives to fill the space between inner balloon catheter 134 and inner proximal stop 300. Although various individual sections of inner proximal stop 300 are shown and described, it should be understood that various other specific examples of sections may be provided, but in many or all examples, a generally ramped surface (or conical or frustoconical geometry) may be provided that increases from outer diameter OD₁ to outer diameter OD₂, with the ramped surface terminating in a shoulder (such as shoulder 308). In some examples, the inner proximal stop 300 may define a central lumen or through bore 314 therethrough, the through bore 314 sized and shaped to snugly fit over the inner balloon catheter 134 during assembly. In embodiments described herein in which ramped (or conical or frustoconical) surfaces are provided, it should be understood that the ramp may be the entire, or only a portion of, the corresponding outer surface.

Now referring in addition to FIGS. 13D-13E, FIGS. 13D-13E show side and rear views, respectively, of an example of distal stop 340 in isolation. In the illustrated example, distal stop 340 may include a plurality of sections, such as two sections. In some examples, a proximal first section 342 may be generally cylindrical, and a plurality of fins 344 may extend radially outward from the first section 342. In some examples, the fins 344 may each extend a distance radially outwardly to define an outer diameter OD₃ of the first section 342. The distal stop 340 in some examples includes a second section 346 distal to the first section 342, the second section 346 tapering to an outer diameter OD₄ that is smaller than outer diameter OD₃. In some examples, the fins 344 extend at least partially along the second section 346, with the fins 344 terminating prior to the distalmost end of the second section 346. In some examples, the distal stop 340 may define a central lumen or through bore 348 therethrough, the through bore 348 sized and shaped to snugly fit over the inner balloon catheter 134 during assembly. In some examples, channels, recesses, or grooves 345 are formed between adjacent ones of the fins 344. In such examples, the grooves 345 may extend along at least part of the length of the first section 342 and at least a part of the length of the second section 346. In some examples, the grooves 345 may enhance the ability of fluid (e.g., inflation media such as saline) to flow around the outside of the distal stop 340.

Referring now in addition to FIG. 13F, in some examples, the balloon 136 may be pulled over the inner balloon catheter 134 after the inner proximal stop 300 and the distal stop 340 have been fixed to the inner balloon catheter 134, and the nose cone 138 may be fixed to the distal end of the inner balloon catheter 134 after the balloon 136 is positioned around the inner balloon catheter 134. However, in other examples, after the inner proximal stop 300 and distal stop 340 are coupled (e.g., fixed, bonded, adhered, etc.) to the inner balloon catheter 134, the proximal end of the inner balloon catheter 134 (not shown in FIG. 13F) is pulled proximally through the distal balloon leg 137b. In these examples, the maximum inner diameter ID₂ of the distal balloon leg 137b may be larger than the maximum outer diameters of the inner proximal stop 300 and distal stop 340, although the maximum inner diameter ID₁ of the proximal balloon leg in these embodiments need not be larger than the maximum outer diameters of the inner proximal stop 300 and distal stop 340. Also, in these examples, the nosecone 138 may be attached to the inner balloon catheter 134 before positioning the balloon 136 over the inner proximal stop 300 and distal stop 340. In the example of FIG. 13F, the inner proximal stop 300 is positioned within proximal pillow 136a, the distal stop 340 is positioned with distal pillow 136b. It should be understood that, in some examples, the balloon 136 is capable of being positioned in FIG. 13F because the inner diameter ID₁ of proximal balloon leg 137a and the inner diameter ID₂ of distal balloon leg 137b are each about equal to or larger than the outer diameter OD₂ of the inner proximal stop 300 and the outer diameter OD₃ of the distal stop 340. It should also be understood that, in the example of the stage of assembly shown in FIG. 13F, the balloon 136 may not yet be fixed to any other components of the delivery system 100.

Referring now in addition to FIGS. 13G-13H, FIGS. 13G-13H are side and front views, respectively, of an example proximal outer stop 320 isolated from other components of the delivery system 100. In some examples, proximal outer stop 320 may include a generally cylindrical proximal rim 322 which may have an annular shape. In some examples, a plurality (e.g., 2, 3, 4, 5, 6 or more) of fingers or extensions 324 may extend distally from the rim 322, the extensions 324 being positioned at spaced distances from each other around a perimeter of the rim 322. In the example shown in FIGS. 13G-13H, the extensions 324 are shown in an expanded condition, although it should be understood that the extensions 324 may be formed so that, in the absence of applied forces, no portions of the extensions 324 extend radially outwardly of an outer diameter OD₅ of the rim 322. In some examples, the extensions 324 may be hingedly coupled to the rim 322, for example via living hinges 326. In some examples, as best shown in the example of FIG. 13H, one or more (including all) of the extensions 324 may include a radially-inwardly extending prong 328 near a distal end thereof.

Referring now in addition to FIGS. 13I-13J, FIGS. 13I-13J are side and front views, respectively, of the proximal outer stop 320 of FIGS. 13G-13H having been assembled to the proximal inner stop 300 of FIGS. 13B-13C, with the assembly isolated from other components of the delivery system 100. As noted above, in some examples, the extensions 324 of the proximal outer stop 320 do not flex outwardly in the absence of applied forces. In some examples, as the extensions 324 of the proximal outer stop 320 are advanced distally over the proximal end of proximal inner stop 300, as the extensions 324 ride along the outer ramped surface of the proximal inner stop 300, the extension 324 flex outwardly via bending at hinge 326. Thus, in this example, as the proximal outer stop 320 continues being advanced over the proximal inner stop 300, the extensions 324 flex farther and farther outwardly until the prongs 328 move past the shoulder 308, at which point the extensions 324 may “snap” back since the extensions 324 are biased to a non-expanded (or collapsed) configuration. As best shown in the example of FIG. 13J, once the prongs 328 engage the shoulder 308, the proximal outer stop 320 is prevented from being pulled proximally relative to the proximal inner stop 300. Further, in some examples, the rim 322 is unable to change size or shape, so that it can only be advanced a given distance distally along the outer ramped surface of the inner proximal stop 300. Thus, in the example shown in FIGS. 13I-13J, once the proximal outer stop 320 has been assembled to the proximal inner stop 300 with the prongs 328 “snapped” over the shoulder 308, the proximal outer stop 320 is substantially axially fixed with respect to the proximal inner stop 300. When in this assembled condition, in some examples, the distal ends of the extensions 324 are positioned along a generally circular profile having an outer diameter OD₆ that is larger than outer diameters OD₅ and OD₂ and also larger than the inner diameter ID₁ of the proximal balloon leg 137a and the inner diameter ID₂ of the distal balloon leg 137b.

Referring now in addition to FIG. 13K, FIG. 13K illustrates the view of FIG. 13F after the proximal outer stop 320 has been slid distally through the interior of the proximal balloon leg 137a and expanded after it slides over and is assembled to the proximal inner stop 300. However, in some examples, including those in which the inner balloon catheter 134 is pulled proximally through the distal balloon leg 137b during assembly, the proximal outer stop 320 may be slid over the proximal end of the inner balloon catheter 134 and positioned just proximal to the proximal inner stop 300, with that assembly being passed proximally through the distal balloon leg 137b. The inner balloon catheter 134 may continue to slide proximally through the proximal outer stop 320 until the nosecone 138 bottoms out on the distal balloon leg 137b. In some examples, the proximal outer stop 320 requires a force applied on its proximal face to force it to advance over the proximal inner stop 300 until the extensions 324 of the proximal outer stop 320 snap over the proximal inner stop 300 (e.g., the shoulder 308 thereof) into place. Referring back to FIG. 13F, prior to passing the proximal outer stop 320 into the interior of the balloon 136, the proximal leg 137a of the balloon 136 remains unfixed to the outer balloon catheter 132. It should be understood that, although the outer balloon catheter 132 is illustrated in FIGS. 13A, 13F, and 13K, the outer balloon catheter 132 may be positioned over the inner balloon catheter 134 in some examples only after the proximal outer stop 320 has been assembled to the proximal inner stop 300 within the balloon 136.

In some examples, after the balloon 136 is positioned around the distal stop 340 and the assembled proximal inner stop 300 and outer stop 320, the outer balloon catheter 132 may be advanced through the interior of the proximal balloon leg 137a until the distal end of the outer balloon catheter 132 is snugly received within the interior of rim 322. In some examples, the distal end of the outer balloon catheter 132 may be fixed to the rim 322, for example via adhesives. In some examples, the distal end of the outer balloon catheter 132 may be fixed to the rim 322 prior the positioning the proximal outer stop 320 within the balloon 136, and in such examples the proximal outer stop 320 may be advanced into the balloon 136 by advancing the outer balloon catheter 132 toward and/or into the balloon 136. In some examples, after the outer balloon catheter 132 is fixed to the proximal outer stop 320, the proximal leg 137a of the balloon 136 may be fixed to the balloon outer catheter 132, for example via adhesive, at a spaced distance proximal to the apertures 132a. In some examples, the distal leg 137b of the balloon 136 may be fixed to the nosecone 138, for example via adhesive. As should be understood, in some examples, by inserting the proximal outer stop 320 into the balloon 136 while it is in a collapsed or unexpanded state, the proximal outer stop 320 is able to fit within the balloon 136. Further, in some examples, by allowing the proximal outer stop 320 to expand only after it has entered the balloon 136, the proximal outer stop 320 may be able to achieve an outer diameter OD₆ that is larger than (e.g., between 1.5 and 2.5 times larger, including about twice as large) the inner diameter ID₁ of the proximal balloon leg 137a and larger than (e.g., between 1.5 and 2.5 times larger, including about twice as large) the inner diameter ID₂ of the distal balloon leg 137b. It should be understood that, in this example, the proximal stop generally (and the proximal outer stop 320 in particular) is able to achieve an outer diameter OD₆ within the balloon 136 that may not be possible if the outer stop 320 had a fixed outer diameter. In some examples, this larger outer diameter may be useful by providing a larger surface against which the prosthetic heart valve 10 may abut, which may provide for additional stability compared to if the prosthetic heart valve 10 abutted a stop having a relatively small outer diameter.

In the examples described in connection with FIGS. 13A-13K, the distal stop 340 is different than the proximal stop and has an outer diameter OD₃ that is smaller than the outer diameter OD₆ of proximal outer stop 320 when it is assembled to proximal inner stop 300. In some examples, it is more important to stabilize the outflow edge of the prosthetic heart valve 10 (which abuts proximal outer stop 320) compared to the inflow edge of the prosthetic heart valve 10 (which abuts distal stop 340), for example because the forward pushing of the delivery assembly 100 (including prosthetic heart valve 10) tends to create proximally-directed forces on the prosthetic heart valve 10 that may need to be counteracted. However, it should be understood that in some examples, the positioning of the proximal stop (i.e., assembled proximal inner stop 300 and proximal outer stop 320) and distal stop 340 may be reversed, while in other examples two versions of distal stop 340 (albeit with orientations that are opposite to each other) may be used, or two versions of the proximal stop (again with orientation that are opposite to each other) may be used. Further, although the examples of FIG. 13K shows the outer balloon catheter 132 extending a distance into the interior of the balloon 136, in other examples little or no length of the outer balloon catheter 132 may extend into the interior of the balloon 136.

Now referring in addition to FIG. 13L, FIG. 13L is a flowchart of example methods of assembling a distal end of a delivery catheter (e.g., delivery catheter 130) that may include one or more of the following steps. In the example method shown, in step 380 a distal stop (e.g., distal stop 340) is affixed to an inner balloon catheter (e.g., inner balloon catheter 134), and in step 382 a proximal inner stop (e.g., proximal inner stop 300) is affixed to the inner balloon catheter. In some examples, steps 380 and 382 may be performed simultaneously, for example if the distal stop and proximal inner stop are formed integrally with the inner balloon catheter via injection molding. In example step 384, a formed balloon (e.g., balloon 136) is positioned over the inner balloon catheter, the distal stop, and the proximal inner stop. In example step 386, a proximal outer stop (e.g., proximal outer stop 320) slides over the inner balloon catheter through the interior of the balloon while the proximal outer stop is in a collapsed condition in the absence of applied forces. In some examples, steps 384 and step 386 are performed by positioning the proximal outer stop over the inner balloon catheter just proximal to the proximal inner stop, and then pulling that entire assembly proximally through a distal leg (e.g., distal balloon leg 137b) of the balloon until a proximal balloon pillow (e.g., proximal pillow 136a) is positioned over the proximal inner stop and proximal outer stop, while a distal balloon pillow (e.g., distal pillow 136b) is positioned over the distal stop. In example step 388, once the proximal outer stop is within the balloon, it is advanced over the proximal inner stop causing the proximal outer stop to expand in diameter, and the proximal outer stop is engaged with the proximal inner stop. In example step 390, an atraumatic distal tip or nose cone (e.g., nose cone 138) is fixed to the distal end of the inner balloon catheter. However, it should be understood that in some embodiments the nosecone may be fixed to the distal end of the inner balloon catheter at, or prior to, the beginning of the assembly process. In an example final step 392, the proximal end of the balloon is fixed to a balloon outer catheter (e.g., balloon outer catheter 132) which at least partially surrounds the balloon inner catheter, and the distal end of the balloon is fixed to the nose cone. It should be understood that the method steps described in connection with FIG. 13L are only exemplary, and more or fewer steps than shown may be performed, and steps may be performed in different orders than shown in FIG. 13L.

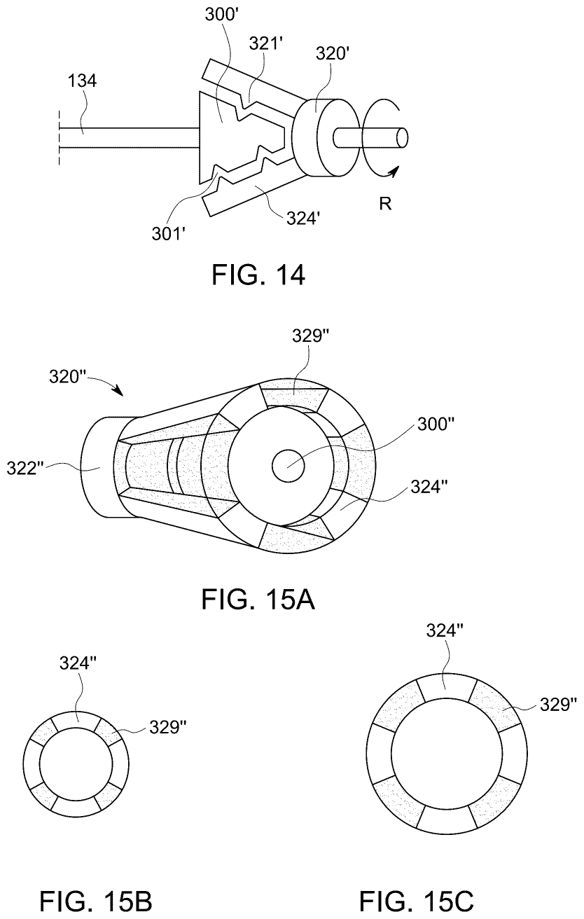

Now referring in addition to FIG. 14, FIG. 14 is a highly schematic side view of an example of a proximal inner stop 300′ assembled to a proximal outer stop 320′ over inner balloon catheter 134. Proximal inner stop 300′ and proximal outer stop 320′ may be similar or identical to counterpart proximal inner stop 300 and proximal outer stop 320, and may in some examples be used in the same way as described in connection with proximal inner stop 300 and proximal outer stop 320, except for the differences explicitly described below. In the example illustrated in FIG. 14, the proximal inner stop 300′ includes helical threads and/or a helically-extending recess 301′ and the extensions 324′ include interiorly-extending protrusions 321′ that have helical positioning corresponding to helically-extending recess 301′. As with proximal outer stop 320, proximal outer stop 300 in some examples may be formed so that the extensions 324′ are biased to a collapsed, non-extended, or small-diameter configuration. However, while in some examples proximal outer stop 320 is pushed and/or translated distally over proximal inner stop 300 during assembly, proximal outer stop 320′ in some examples may be rotated (e.g., in rotational direction R) relative to proximal inner stop 300′. In these examples, the protrusions 321′ are received within the recess 301′, and as the proximal outer stop 320′ is rotated relative to proximal inner stop 300′, the proximal outer stop 320′ is pulled distally as the threading/rotating continues, forcing the extensions 324′ to expand as they drive forward over the outer ramped surface of the proximal inner stop 300′. Other than the threading feature described herein, the structure and use of the example of FIG. 14 may be similar or identical to the features and methods described in connection with FIGS. 13A-13L. It should be understood that, due to threading function and structure, in some examples, the shoulder 308 described in connection with inner proximal stop 300, and the prongs 328 described in connection with outer proximal stop 320, may be omitted from the example shown in FIG. 14.

Now referring in addition to FIG. 15A, FIG. 15A is a highly schematic side view of an example of a proximal inner stop 300″ assembled to a proximal outer stop 320″. Proximal outer stop 320″ may be similar or identical to either proximal outer stop 320 or proximal outer stop 320′, with the exception of material 329″ described in greater detail below. Proximal inner stop 300″ may be similar or identical to proximal inner stop 300 or proximal inner stop 300′. Thus, the similar features are not described in detail again here. In the example shown in FIG. 15A, a material 329″ is connected circumferentially between each adjacent pair of extensions 324″ and to the rim 322″. In some examples, the material 329″ is provided between all of the pairs of extensions 324″ so that the extensions 324″ and material 329″, in combination, form a substantially continuous surface. In some examples, the material 329″ may be soft and may be formed of an elastic material capable of stretching. In some examples, the material 329″ effectively connects each extension 324″ to each other extension 324″, even if such connection is indirect. Now referring in addition to FIG. 15B, FIG. 15B is a highly schematic cross-section of the proximal outer stop 320″ in an initial or collapsed condition, for example prior to being advanced over (and/or assembled to) the proximal inner stop 300″. As the proximal outer stop 320″ is advanced over the proximal inner stop 300″ (e.g., via pushing as shown and described in connection with FIGS. 13A-13L or by rotating as shown and described in connection with FIG. 14), the extensions 324″ may begin to flare outwardly. However, in some examples, because the material 329″ connects adjacent pairs of extensions 324″ along some, most, or all of the length of the extensions 324″, the material 329″ (which as noted above may be elastic) may cause the extensions 324″ to flare outwardly in a more radially symmetric fashion than might occur in the absence of the material 329″. Now referring in addition to FIG. 15C, FIG. 15C is a highly schematic cross-section of the proximal outer stop 320″ in an assembled or expanded condition, for example after being advanced over (and/or assembled to) the proximal inner stop 300″ (which is omitted from the view of FIG. 15C for clarity of illustration). As can be seen in the example of FIG. 15C, the material 329″ may help the extensions 324″ in maintaining a circular and/or radially symmetric profile during and after expansion. In some examples, because the material 329″ may eliminate any spacing between adjacent pairs of extensions 324″, a softer and/or more consistent interface may be provided against which the balloon 136 may contact, which may reduce potential damage (e.g., pinching between adjacent extensions 324″) to the balloon 136. Other than the material 329″ described herein, the structure and use of the example of FIGS. 15A-15C may be similar or identical to the features and methods described in connection with FIGS. 13A-13L and/or FIG. 14.

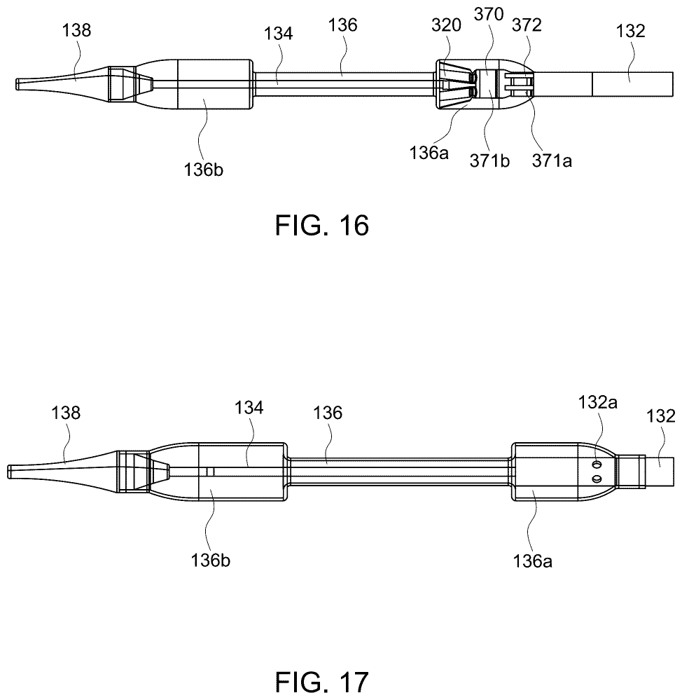

In the examples described in connection with FIGS. 13A-13L, including in particular the view of FIG. 13K, the outer balloon catheter 132 extends into the proximal pillow 136 and contacts and/or is fixed to the proximal outer stop 320. In these types of examples, the outer balloon catheter 132 may provide additional column strength to the proximal outer stop 320, which may be desirable. However, as noted above, in some examples the outer balloon catheter 132 does not extend into (or does not extend a significant distance into) the interior of the proximal pillow 136a. Now referring in addition to FIG. 16, FIG. 16 shows an example that is similar to the assembly of FIG. 13K but in which the outer balloon catheter 132 does not extend into the interior of the proximal pillow 136a. In this particular example, in order to provide enhanced column strength to the assembly, a pusher 370 is included between the distal end of the outer balloon catheter 132 and the rim 322 of the outer proximal stop 320. In some examples, the proximal end of the pusher 370 may be in abutting contact with and/or fixed to the distal end of the outer balloon catheter 132, and the distal end of the pusher 370 may be in abutting contact with and/or fixed to the proximal end of the outer proximal stop 320 (e.g., the rim 322). In the absence of the pusher 370, in examples in which the outer balloon catheter 132 does not extend into the proximal pillow 136a, a potential problem could arise. In such an example, as the delivery catheter 130 is advanced through the patient's vasculature, the force from the outer balloon catheter 132 being pushed distally could cause the proximal pillow 136a to collapse and/or buckle. However, in the example of FIG. 16, the pusher 370, which may be a substantially rigid structure, may provide column strength between the outer balloon catheter 132 and the proximal outer stop 320, preventing or reducing the likelihood of buckling of the proximal pillow 136a during use. In some examples, the pusher 370 may include a generally cylindrical a proximal section 371a that had an outer diameter about equal to or slightly larger or smaller than the outer diameter of the outer balloon catheter 132. In some examples, the pusher 370 may include a generally cylindrical distal section 371b that has an outer diameter that is larger than that of the proximal section 371a, and which is configured to abut the rim 322, be received within the rim 322, or be received over the rim 322. In some examples, the proximal section 371a includes one or more axially extending slots 372 which may create channels that are in fluid communication with the interior of the outer balloon catheter 132. In such examples, during de-airing, deflation, and/or inflation of the balloon 136, fluid may pass from the interior of the outer balloon catheter 132, through the channels of the slots 372, and into the proximal pillow 136a. In some examples, if slots 372 are provided, they may be provided at substantially equal intervals around the circumference of the proximal section 371a. In some examples, if slots 372 are included, they may extend any desired length along the pusher 370 that provides for fluid communication between the interior of the outer balloon catheter 132 and the interior of the proximal pillow 136a.

Now referring in addition to FIG. 17, FIG. 17 is a side view of an example of the distal end of delivery catheter 130 in which the outer balloon catheter extends into the interior of proximal pillow 136a, but additional stop features or valve retainers are not provided. In the example of FIG. 17, the outer balloon catheter 132 extends most or all (e.g., at least 80%, at least 90%, or about 100%) of the axial distance of the proximal pillow 136a. In the illustrated example, the distalmost end of the outer balloon catheter 132 terminates at about the same location, in the axial direction, that the proximal pillow 136a terminates. In this example, although a separate valve retainer or stop is not provided, the distal end of the outer balloon catheter 132 may functionally serve as a proximal stop, although the outer balloon catheter 132 does not have as large an outer diameter as certain other examples described herein, for example including outer diameter OD₆ of proximal inner stop 320 which may be a larger diameter than outer balloon catheter 132. However, even with the outer balloon catheter 132 having a relatively small outer diameter in this example, it may still provide a suitable level of axial force against the outflow end of the prosthetic heart valve 10 (some of which force may be transmitted through the distal end of the proximal pillow 136a) when the prosthetic heart valve 10 is crimped over the balloon 136 with the terminal ends of the prosthetic heart valve 10 abutting the proximal pillow 136a and the distal pillow 136b. As noted in connection with FIGS. 13A-L, one or more apertures 132a may be provided in the wall of the outer balloon catheter 132, with the apertures 132a being located at or near a proximal end of the proximal pillow 136a and in fluid communication with the interior of the proximal pillow 136a. In some examples, the apertures 132a are provided at substantially equal intervals around the circumference of the outer balloon catheter 132, for example four apertures 132a provided every 90 degrees (+/−10 degrees), five apertures 132a provided every 72 degrees (+/−10 degrees), etc. In use, at any point at which fluid is being introduced into the interior of the balloon 136 or removed from the interior of the balloon 136, the fluid flows through a pathway between the inner balloon catheter 134 and the outer balloon catheter 132. However, in examples in which the outer balloon catheter 132 extends a distance into the interior of the balloon 136, the point at which fluid enters or exits the balloon may be a spaced distance from the proximal end of the balloon 136. For example, in the example of FIG. 17, if apertures 132a were omitted, fluid flowing into or out of the balloon 136 would enter/exit near the distal end of the proximal pillow 136a. This may not be desirable in all circumstances. For example, it may be desirable during inflation of the balloon 136 for fluid to easily enter the proximal pillow 136a. Further, during de-airing of the balloon 136 when the delivery system 100 is being prepared for use, in the absence of apertures 132a, air bubbles may tend to get trapped within proximal pillow 136a, including near the point at which the proximal pillow 136a (or proximal leg 137a) is fixed to the outer balloon catheter 132. However, by providing one or more apertures 132a that provide an additional inlet/outlet for fluid flowing between the outer balloon catheter 132 and balloon 136, inflation media may tend to more easily enter the proximal pillow 136a during inflation and exit the proximal pillow 136a during deflation, while air bubbles that might otherwise get trapped within the proximal pillow 136a may more easily be removed through the system via apertures 132a during de-airing. It should be understood that the description of the structure and function of apertures 132a described in connection with FIG. 17 may be applied to all other embodiments herein in which the outer balloon catheter 132 extends a distance into the interior of the balloon 136 (including for example the examples of FIGS. 13A-13L). Further, in some examples, the entire outer balloon catheter 132, or only the portion extending into the proximal pillow 136a, may be formed of Pebax 6333, or another polyether block amide, having a Shore durometer of about 63 D. However, in other examples, the entire outer balloon catheter 132, or only the portion extending into the proximal pillow 136a, may be formed of a polyether block amide or nylon having a Shore durometer of between about 55 D and about 90 D, which may provide the desired column strength without being too stiff in axial bending.

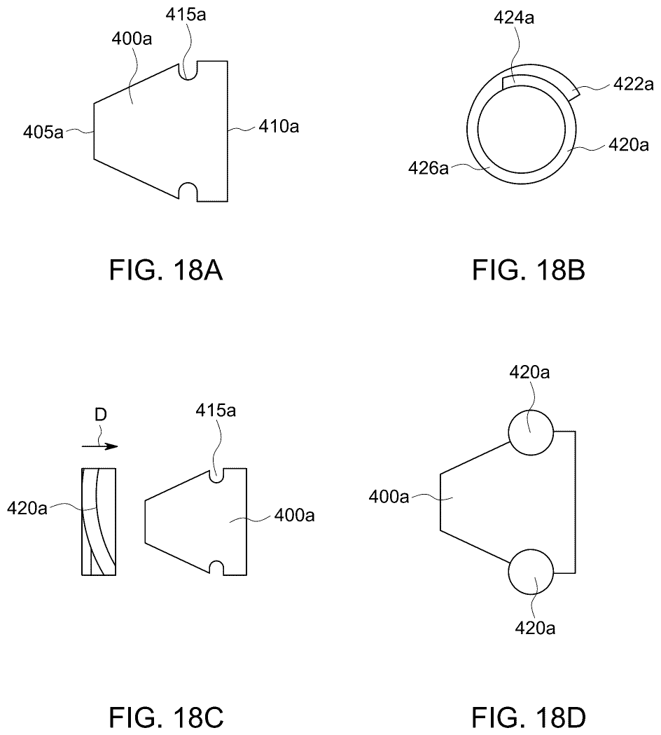

Now referring in addition to FIG. 18A, FIG. 18A is a schematic side view of an example of an inner stop 400a that may be used as part of an expanding proximal and/or distal stop or valve retainer in a similar fashion as the proximal stop(s) described in connection with FIGS. 13A-15C. Inner stop 400a in some examples may have a generally conical or frustoconical shape extending between a first end 405a having a relatively small outer diameter and a second end 410a having a relatively large outer diameter. If inner stop 400a is being used as a proximal stop, first end 405a in some examples may be the proximal end and second end 410a may be the distal end. If inner stop 400a is being used as a distal stop, first end 405a in some examples may be the distal end and second end 410a may be the proximal end. In some examples inner stop 400a may include a circumferential recess 415a sized and shaped to receive an outer stop 420a therein. If recess 415a is included, in some examples it is positioned nearer the second end 410a than the first end 405a where the outer diameter of the inner stop is relatively large. Although not shown in FIG. 18A, inner stop 400a may include a through bore (such as through bore 348) so that the inner stop 400a may be slid over and fixed (e.g., via adhesives) to inner balloon catheter 134. However, in other embodiments, inner stop 400a may be formed integrally with and/or overmolded onto inner balloon catheter 134.

Now referring in addition to FIG. 18B, FIG. 18B is a schematic front view of an example of an outer stop 420a that may be used with inner stop 400a. Outer stop 420a in the illustrated example is a coil having a first end 422a, a second end 424a, and an intermediate portion 426a extending between the first end 422a and the second 424a. The intermediate portion 426 in some examples includes one or more loops forming a generally circular shape, and in the absence of applied forces, the outer stop 420a may have an inner diameter that is larger than the outer diameter of the first end 405a but smaller than the outer diameter of the second end 410a of the inner stop 400a.

To assemble the distal end of the delivery catheter 130, the maximum outer diameter of the inner stop 400a (e.g., at the second end 410a) may in some examples be small enough to fit through either proximal balloon leg 137a or distal balloon leg 137b. Similarly, in the absence of applied forces, the outer diameter of the outer stop 420a may in some examples be small enough to fit through either proximal balloon leg 137a or distal balloon leg 137b. Once the balloon 136 is positioned over both the inner stop 400a and the outer stop 420a, in the example shown in FIG. 18C, the outer stop 420a may be advanced in direction D toward the first end 405a of the inner stop 400a. If the stop is being used as a proximal stop, the direction D may be the distal direction, and if the stop is being used as a distal stop, the direction D may be the proximal direction. In some examples, as the outer stop 420a is advanced along the inner stop 400a, the ramped outer surface of the inner stop 400a forces the outer stop 420a to expand in diameter as the outer stop 420a begins to uncoil (e.g., the first end 422a and second end 424a may begin to move closer to each other as the amount of overlap between the first end 422a and the second end 424a decreases). In some examples the outer stop 420a may continue to translate along the ramped surface of the inner stop 400a toward the second end 410a until the outer stop 420a reaches the recess 415a. Now referring in addition to FIG. 18D, FIG. 18D is a highly schematic cross-section of the configuration in which the outer stop 420a has been advanced along the inner stop 400a until the outer stop 420a has reached the recess 415a and “snapped” back into the recess 415a at least in part due to the outer stop 420a being biased to a small diameter configuration. In the resulting configuration of the example of FIG. 18D, the outer stop 420a is effectively locked to the inner stop 400a due to the engagement with the recess 415a, and the outer stop 420a has an outer diameter that is larger than the largest outer diameter of the inner stop 400a. In other words, in some examples, similar to the examples described in connection with FIGS. 13A-13L, the assembled stop (including inner stop 400a and outer stop 420a) can have a larger outer diameter than the maximum size that can readily be inserted through either proximal balloon leg 137a or distal balloon leg 137b, providing for a relatively large diameter stop surface for assisting with retaining the axial position of the prosthetic heart valve 10. It should be understood that, in terms of positioning with respect to the remainder of the delivery system 100, inner stop 400a and outer stop 420b, when assembled, may have the same or similar position as the proximal inner stop 300 and proximal outer stop 320 (if being used as a proximal stop) or the same or similar position as the distal stop 340 (if being used as a distal stop) as shown in the example of FIG. 13K.

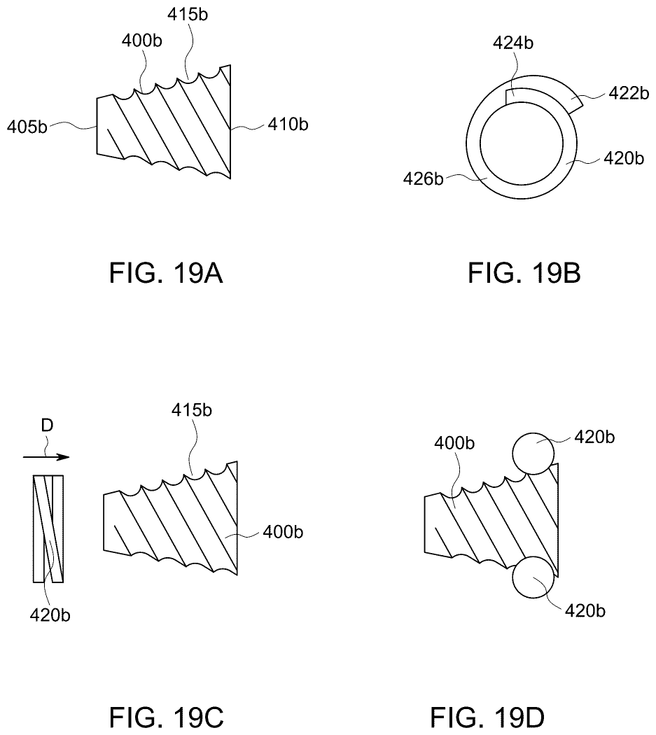

Now referring in addition to FIGS. 19A-19D, FIGS. 19A-19D illustrate an example of an inner stop 400b and an outer stop 420b which are similar (in both configuration and use) to the example inner stop 400a and outer stop 420b of FIGS. 18A-18D. For example, inner stop 400b may be ramped from first end 405b to second end 410b in substantially the same fashion as inner stop 400a. However, in this example, the main difference between inner stop 400a and inner stop 400b is that, instead of including a circumferential recess 415a like stop 400a, inner stop 400b includes a helical recess or thread 415b extending along most or all of the length of the inner stop 400b. As shown in FIG. 19B, the outer stop 420b may be identical to outer stop 420a, and is thus not described in detail again here, other than that the outer stop 420b may include a first end 422b, a second end 424b, an intermediate section 426b extending between the ends, and that the outer stop 420b may be biased to a relatively small diameter in the absence of applied forces.

Now referring in addition to FIG. 19C, FIG. 19C shows that in some examples the outer stop 420b may be assembled to the inner stop 400b (e.g., after the balloon 136 has been positioned over both stops) in a generally similar fashion as shown and described in connection with FIG. 18C. However, whereas in the example of FIG. 18C, the outer stop 420a is pushed over the inner stop 400a in direction D, in the example of FIG. 19C, the outer stop 420b is advanced in direction D over the inner stop 400b by rotating the outer stop 420b relative to the inner stop 400b so that the outer stop 420b effectively threads over the helical recess or thread 415b. Similar to as described in connection with the example of FIG. 18C, as the outer stop 420b advances toward the second end 410b, it will increase in diameter as the outer stop 420b unwinds or uncoils. Referring now in addition to FIG. 19D, similar as described in connection with FIG. 18D, when the outer stop 420b is assembled to the inner stop 420a, the outer stop 420b may have an outer diameter that is larger than the largest outer diameter of the inner stop 400b. Otherwise, the functionality described in connection with inner stop 400a and outer stop 420a may be similar or identical to the functionality of inner stop 400b and outer stop 420b, and is thus not described in further detail again here.

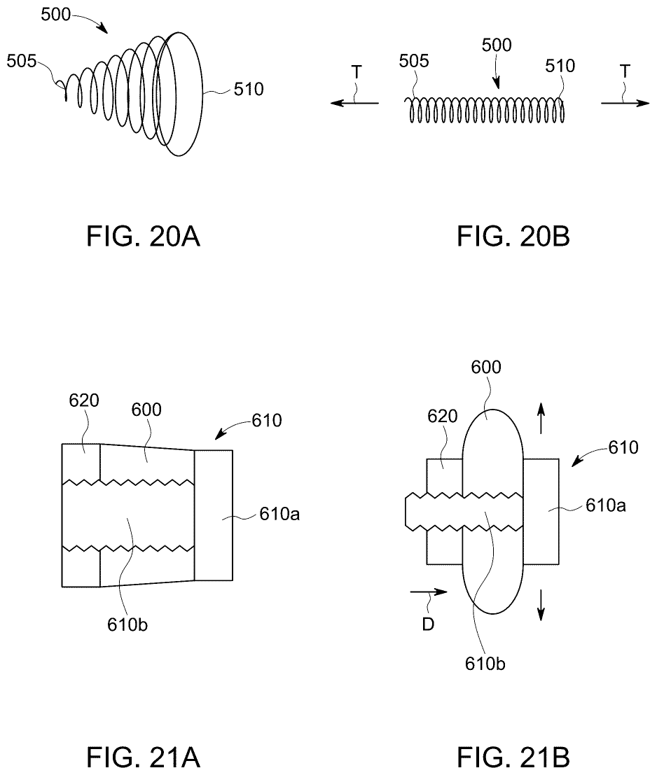

Now referring in addition to FIG. 20A, FIG. 20A is a highly schematic illustration of an example of a stop 500 in a relaxed state (e.g., in the absence of applied forces). In the illustrated example, the stop 500 is formed of a coiled wire (which may in some examples be a single wire and in other examples by formed by two or more wire strands braided together) that extends from a first end 505 to a second end 510. In the illustrated example, the coiled wire that forms stop 500 is biased so that, when the stop 500 is relaxed, the second end 510 has a larger outer diameter than the first end 505. For example, the second end 510 may have an outer diameter when relaxed that is larger than the inner diameters of the proximal balloon leg 137a and the distal balloon leg 137b. In some examples, to assemble the distal end of the delivery catheter 130, after the balloon 136 is positioned over the inner balloon catheter 134, the first end 505 of stop 500 may be pulled away from the second end 510 of stop 500 in opposite directions of tension T to temporarily force the stop 500 to have a reduced diameter in which the maximum outer diameter of the stop 500 is small enough to readily fit through either the proximal balloon leg 137a and/or the distal balloon leg 137b. An example of this temporarily tensioned and reduced-diameter state is shown in FIG. 20B. While the stop 500 has a temporary configuration similar to the example shown in FIG. 20B, it may be passed over the inner balloon catheter 134 and through either the proximal balloon leg 137a or distal balloon leg 137b into the proximal pillow 136a or the distal pillow 136b. In this example, once the stop 500 is within the proximal pillow 136a or the distal pillow 136b, the tension on the stop 500 may be released, which may allow the stop 500 to return mostly or entirely to the example shape shown in FIG. 20A.

Similar to the examples of the stops shown and described in connection with FIGS. 18A-19D, stop 500 may be used as a proximal stop, a distal stop, or both. However, dissimilar to the examples shown and described in connection with FIGS. 18A-19D, stop 500 may be used without a separate inner and outer component, at least in part because the stop 500 is set to the larger diameter when unbiased, whereas the outer stops 420a, 420b in the examples of FIGS. 18A-19D are set to a smaller diameter when unbiased. In some examples, it may be useful to provide additional stiffness to stop 500 because it may lack a separate inner component to which it may be fixed. For example, in some examples the stop 500 may be formed of a nickel-titanium alloy (e.g., nitinol), and shape-set to a relatively stiff austenite phase at or around typical body temperature. In such examples, when the stop 500 is used within a patient, it may increase in temperature to or close to the patient's body temperature, and become stiffer within the body than when it is outside of the body, which may provide for enhanced valve retention.

Now referring in addition to FIG. 21A, FIG. 21A is a schematic cross-section of another example of a stop 600. In the illustrated example, stop 600 may be formed of a compressible material and have a generally cylindrical shape with an interior through bore configured to be received over a shaft 610b of a bolt 610. Bolt 610 in the illustrated example may also include a head 610a coupled to the shaft 610b. In some examples, bolt 610 may be provided as a unit that can be slid over and coupled to the inner balloon catheter 134, for example at a position over which the proximal pillow 136a or distal pillow 136b will eventually surround. In some examples, the bolt 610 may be fixed (e.g., via adhesives) or be formed integrally with (e.g., by injection molding or overmolding) the inner balloon catheter 134. In some examples, the shaft 610b may include exterior threads. In the illustrated example, a nut 620 may be positioned over the shaft 610b, and the nut 620 may include internal threading so that the nut 620 may be threaded over the shaft 610b to translate the nut 620 toward or away from the head 610a. The stop 600 may in some examples be positioned with the opposite terminal ends of the stop 600 abutting the head 610a of the bolt 610 and the nut 620. The configuration shown in the example of FIG. 21A may represent an initial state prior to the balloon 136 being pulled over the components shown in FIG. 21A. In this example initial state, the outer diameters of the head 610a, stop 600, and nut 620 may be small enough to readily pass through the proximal leg 137a and/or distal leg 137b of the balloon 136. It should be understood that the example assembly shown in FIG. 21A may be used as a proximal stop within proximal pillow 136a, as a distal stop within distal pillow 136b, or both.

Now referring in addition to FIG. 21B, after the balloon 136 has been positioned over the stop 600 while the stop 600 is in an uncompressed (or substantially uncompressed) condition, the nut 620 may be rotated to drive the nut 620 in a direction D toward the head 610a. In this example, because the stop 600 is formed of a compressible and/or elastic material, as the nut 620 advances along shaft 610b, the nut 620 and head 610a together compress the stop 600 in an axial direction, forcing the stop 600 to expand radially. In one example, the nut 620 may be advanced toward head 610a until the stop 600 has been expanded radially outwardly to a desired diameter. In some examples, the outer diameter of the stop 600 after it has been expanded radially, for example similar to the configuration shown in FIG. 21B, is larger than the inner diameter of the proximal leg 137a and/or distal leg 137b of the balloon. The overall functionality of the example stop 600 shown and described in connection with FIGS. 21A-21B may be generally similar or identical to the other examples of expandable stops described herein, and is thus not described in greater detail here. In some examples, because stop 600 does not rely on any ramped surface to expand, the orientation of the bolt head 610a compared to the nut 620 (e.g., which is positioned proximally of the other) may not be important.

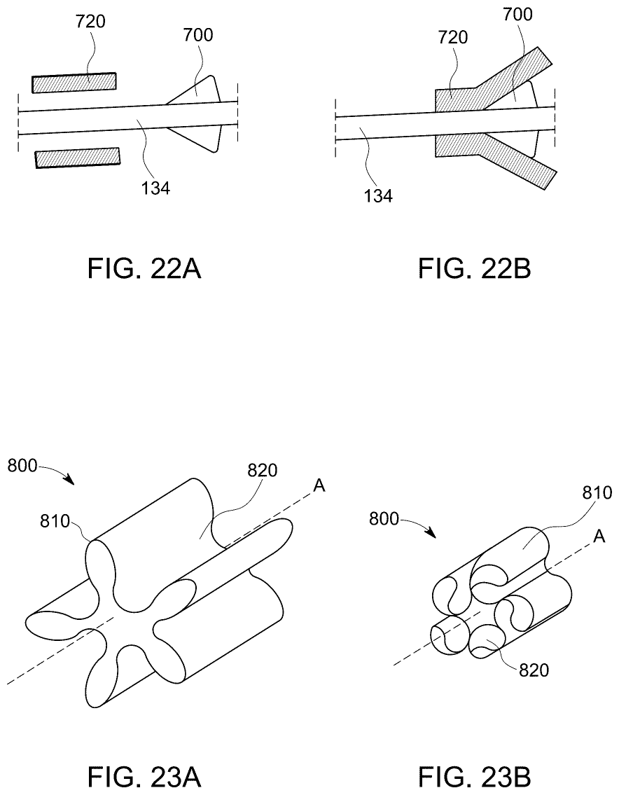

Now referring in addition to FIG. 22A, FIG. 22A is highly schematic cross-section of an example of an inner stop 700 in an initial state of assembly with an outer stop 720. Inner stop 700 may be fixed (e.g., by adhesives) and/or integrally formed (e.g., via injection molding or overmolding) to the inner balloon catheter 134. As with other inner stops described herein (e.g., inner proximal stop 300 or inner stops 400a, 400b), inner stop 700 may be substantially conical or frustoconical with the larger diameter end configured to abut an end of the prosthetic heart valve 10 when the prosthetic heart valve 10 is crimped over balloon 136 between proximal pillow 136a and distal pillow 136b. The example of outer stop 720 in FIG. 22A may be an elastic sleeve, which may for example have a generally cylindrical shape. In some examples, the outer diameter of the outer stop 720 may be about equal to or smaller than the largest outer diameter of the inner stop 700 when no forces are applied to the outer stop 720. As with other examples described herein, the largest outer diameters of the outer stop 720 (in the absence of applied forces) and of the inner stop 700 may in some examples be about equal or slightly smaller than the inner diameter of the proximal balloon leg 137a and/or the distal balloon leg 137b. In such examples, as shown in FIG. 22A, the balloon 136 may readily be slid over the inner stop 700 and outer stop 720 (when the outer stop 720 is in the unbiased configuration). Once the inner stop 700 and the outer stop 720 are both positioned in the proximal pillow 136a or the distal balloon 136b, in some examples the outer stop 720 may be manually advanced over the stop 700. In this example, as shown in FIG. 22B, as the outer stop 720 advances over the ramped outer surface of the inner stop 700, the outer stop 720 expands in diameter, at least in part due to its elasticity. In some examples, the outer stop 720 can be advanced beyond the large-diameter end of the inner stop. In various examples, once the outer stop 720 is assembled to the inner stop 700, for example similar to the configuration shown in FIG. 22B, the largest outer diameter of the outer stop 720 may be larger than the inner diameter of the proximal balloon leg 137a and/or the distal balloon leg 137b. In some examples, the outer stop 720 and/or inner stop 700 may include features to assist with locking of the outer stop 720 to the inner stop 700 in the desired position. For example, in some examples the outer stop 720 may include inwardly facing prongs similar or identical to prongs 328, and the inner stop 700 may include a shoulder similar or identical to shoulder 308. The overall functionality of the example inner stop 700 and outer stop 720 shown and described in connection with FIGS. 22A-22B may be generally similar or identical to the other examples of expandable stops described herein, and is thus not described in greater detail here.

Referring now in addition to FIG. 23A, FIG. 23A shows another example of a stop 800. FIG. 23A illustrates an example of stop 800 in an uncompressed or unbiased condition. In some examples, stop 800 may be formed as an ultra-thin foldable extrusion. In some examples, the ultra-thin foldable extrusion may have a thickness of less than about 0.01 inches (0.254 mm), including between about 0.001 inches (0.0254 mm) and about 0.005 inches (0.127 mm). In some examples, the stop 800 maybe formed of nylon or a high durometer polyether block amide (e.g., the product offered under the tradename Pebax®). In some examples, the material forming the stop 800 may have a durometer of between about 63 D and about 90 D Shore durometer. In the example of FIG. 23A, the stop 800 is formed to extend along a central longitudinal axis A with a hollow interior. The stop 800 in some examples has a star- or flower-shape, for example with a plurality of fins or petals 810. In some examples, each petal 810 may be formed of two wall portions of the stop 800 extending radially outward from near the central longitudinal axis, and circumferentially adjacent petals 810 may form a groove or channel 820 therebetween. The groove or channel 820 may in some examples be formed by a wall portion of the stop that extends radially inwardly toward central longitudinal axis A from the two adjacent petals 810. In some examples, the stop 800 is formed as an extrusion and includes a continuous structure around the central longitudinal axis A. In other examples, the stop 800 is formed as a flat sheet with edges of the flat sheet coupled together and the flat sheet being shaped to have a shape similar to the example of FIG. 23A. In some examples, in the absence of applied forces, the stop 800 has a shape similar to that shown in FIG. 23A. In some examples, the radially-outer-most points on the plurality of petals 810 may be inscribed by a circle having a diameter that is larger than the inner diameter of the proximal leg 137a and/or the inner diameter of the distal leg 137b.

Now referring in addition to FIG. 23B, in order to reduce the size of stop 800 to fit for insertion into the proximal leg 137a or the distal leg 137b, in some examples the petals 810 may be temporarily wrapped or twisted around the central longitudinal axis A. As shown in the example of FIG. 23B, in this wrapped or twisted state, the petals 810 in some examples curl around the central longitudinal axis A instead of extending mostly or exclusively radially outwardly from the central longitudinal axis A. In this example, the outer perimeter of the stop 800 may be inscribed by a circle having a diameter that is smaller than the inner diameter of the proximal leg 137a and/or the inner diameter of the distal leg 137b so that, while in this temporarily reduced-diameter state, the stop 800 may be inserted into the balloon 136 through the proximal leg 137a or the distal leg 137b. It should be understood that, in both states of the examples of FIGS. 23A and 23B, the inner balloon catheter 134 may extend through the interior of the stop 800, for example generally along the central longitudinal axis A of the stop 800. In some examples, after the stop 800 is inserted into the proximal pillow 136a or the distal pillow 136b while in the curled or wrapped condition, the grip on the stop 800 may be released allowing the stop 800 to unwrap or unfurl back toward an undeformed state similar to that shown in FIG. 23A. In some examples, after partly or entirely returning to a shape similar to that shown in FIG. 23A, the large outer diameter of the stop 800 may act to provide a stop surface against which the inflow or outflow end of the prosthetic heart valve 10 may contact.

In some examples, stop 800 may be used in isolation without a separate inner stop as described in connection with other embodiments herein. In such examples, the large surface area of stop 800 may tend to create enough friction so that the stop 800 is not able to move significantly during use, even if forces are applied in the axial direction (e.g., by the prosthetic heart valve 10 pressing on an end of the stop 800) onto the stop 800 during use. The example of stop 800 shown in FIGS. 23A-23B may allow for fluid (e.g., air during de-airing, or saline during balloon deflation or inflation) to easily move through the hollow central section of stop 800 without obstruction. The overall functionality of the example stop 800 shown and described in connection with FIGS. 23A-23B may be generally similar or identical to the other examples of expandable stops described herein, and is thus not described in greater detail here.

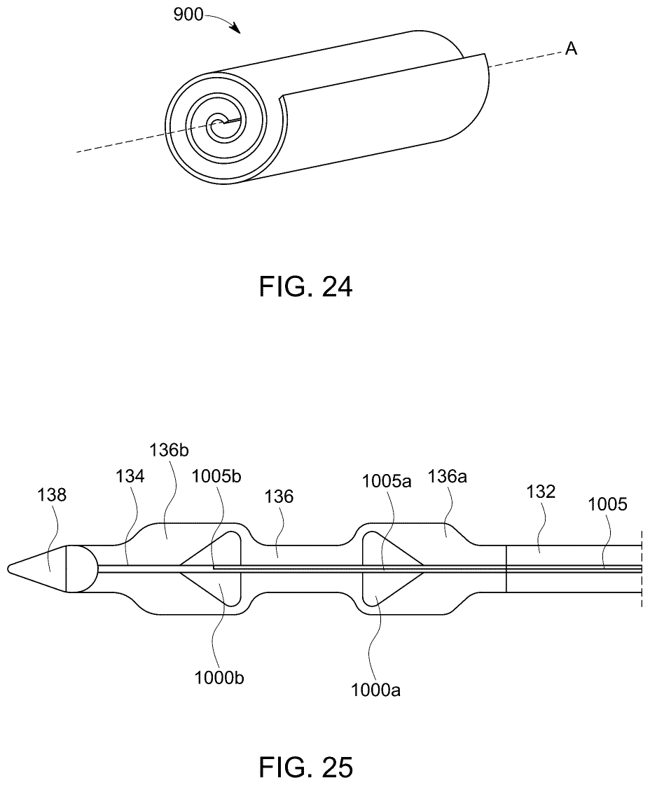

Referring now in addition to FIG. 24, FIG. 24 shows another example of a stop 900. FIG. 24 illustrates an example of stop 900 in an uncompressed or unbiased condition. In some examples, stop 900 may be formed as an extrusion and/or flat sheet that is rolled into a spiral configuration, with stop spiraling around a central longitudinal axis A. Stop 900 in some examples may be formed of a material similar to that described in connection with stop 800 and may be used substantially the same manner. Briefly, when the stop 900 is in an unbiased or unwrapped or uncompressed configuration, the outer diameter of the stop 900 (in a direction orthogonal to the central longitudinal axis A) may in some examples be larger than the inner diameter of the proximal balloon leg 137a and/or the inner diameter of the distal balloon leg 137b. Similar to as described in connection with FIG. 23B, in some examples stop 900 may be temporarily transitioned into a reduced diameter configuration in which the space between adjacent layers of the spiral shape are reduced. In other words, in some examples the stop 900 may be more tightly wound to temporarily reduce the outer diameter of the stop 900, at which point it may be inserted into the interior of the balloon 136 (e.g., through proximal balloon leg 137a or distal balloon leg 137b). In this example, after the stop 900 is inserted into the proximal pillow 136a or the distal pillow 137b, it will begin to unfurl or unroll partly or completely back to the initial configuration, similar to that shown in FIG. 24. Otherwise, in some examples, the functionality of stop 900 may be similar or identical to that described in connection with stop 800, including the ability of fluid to readily flow through the interior of the stop 900 during inflation, deflation, and/or de-airing of the balloon 136.

Now referring in addition to FIG. 25, FIG. 25 illustrates an example of a distal end of delivery catheter 130 which includes inflatable stops 1000a, 1000b. In the illustrated example, a proximal stop 1000a is provided within the proximal pillow 136a and a distal stop 1000b is provided within the distal pillow 136b. However, it should be understood that proximal stop 1000a may be provided in some examples without a corresponding distal stop 1000b, and in other examples the distal stop 1000b may be provided without a corresponding proximal stop 1000a. In the illustrated example, each stop 1000a, 1000b is formed of an inflatable member, such as a secondary balloon positioned within balloon 136. In the illustrated example, proximal stop 1000a may have an inflated or expanded condition in which the proximal stop 1000a has a generally conical or frustoconical shape, with the smaller diameter end of the proximal stop 1000a positioned proximally and the larger diameter end of the proximal stop 1000a positioned distally, in substantial alignment with or proximally adjacent to the distal end of the proximal pillow 136a. Similarly, in the illustrated example, distal stop 1000b may have an inflated or expanded condition in which the distal stop 1000b has a generally conical or frustoconical shape, with the smaller diameter end of the distal stop 1000b positioned distally and the larger diameter end of the distal stop 1000b positioned proximally, in substantial alignment with or distally adjacent to the proximal end of the distal pillow 136b.

Still referring to FIG. 25, in some examples during manufacturing the proximal stop 1000a and/or distal stop 1000b may be coupled to the inner balloon catheter 134 and in a deflated condition before positioning the balloon 136 over the proximal stop 1000a and/or distal stop 1000b. In some examples, after the balloon 136 is positioned over the proximal stop 1000a and/or distal stop 1000b, the proximal stop 1000a and/or distal stop 1000b may be expanded by filling with a settable material, such as filling with epoxy that hardens, for example taking the shape shown in FIG. 25. In the example of FIG. 25, when the proximal stop 1000a and the distal stop 1000b are inflated or expanded, the outer diameter of the larger diameter end of each stop 1000a, 1000b may be larger than the inner diameter of the proximal leg 137a and/or the inner diameter of the distal leg 137b. With this type of embodiment, the proximal stop 1000a and distal stop 1000b may be permanently inflated after manufacturing and not need to be inflated again. If the proximal stop 1000a and distal stop 1000b are inflated with epoxy or a similar hard-setting fluid during the manufacturing stage, the filling may be performed while the balloon 136 surrounds the proximal stop 1000a and/or distal stop 1000b, but before the balloon 136 is fixed to the nose cone 138 and/or to the outer balloon catheter 132. This example of manufacturing may provide for additional filling options as the proximal stop 1000a and distal stop 1000b may be directly accessible through the proximal balloon leg 137a and/or the distal balloon leg 137b prior to the balloon 136 being fixed to other components of the delivery catheter 130.

In some embodiments, the proximal stop 1000a and distal stop 1000b may be configured to be inflated, filled, and/or expanded by the user during preparation for implantation of prosthetic heart valve 10. In such examples, one or more separate inflation lumens (e.g., inflation lumen 1005) may be provided to couple a fluid reservoir (e.g., a syringe similar to syringe 174) to the interior of the proximal stop 1000a and distal stop 1000b. In some examples, these separate inflation lumen(s) 1005 may run through the wall of the inner balloon catheter 134 with openings (e.g., openings 1005a, 1005b) to the interior of the proximal stop 1000a and/or the distal stop 1000b. In this type of example, the proximal stop 1000a and distal stop 1000b may be inflated or expanded just prior to crimping the prosthetic heart valve 10 onto the balloon 136. Similarly, in this type of example, the proximal stop 1000a and/or distal stop 1000b may be deflated at any desired point in the procedure, for example immediately after the prosthetic heart valve 10 is deployed. Whether the proximal stop 1000a and/or distal stop 1000b are expanded during manufacturing with a fluid that sets into a solid or is filled with saline (or another non-setting fluid) just prior to use, the inflated/expanded proximal stop 1000a and/or distal stop 1000b may be substantially rigid valve retention structures upon inflation.

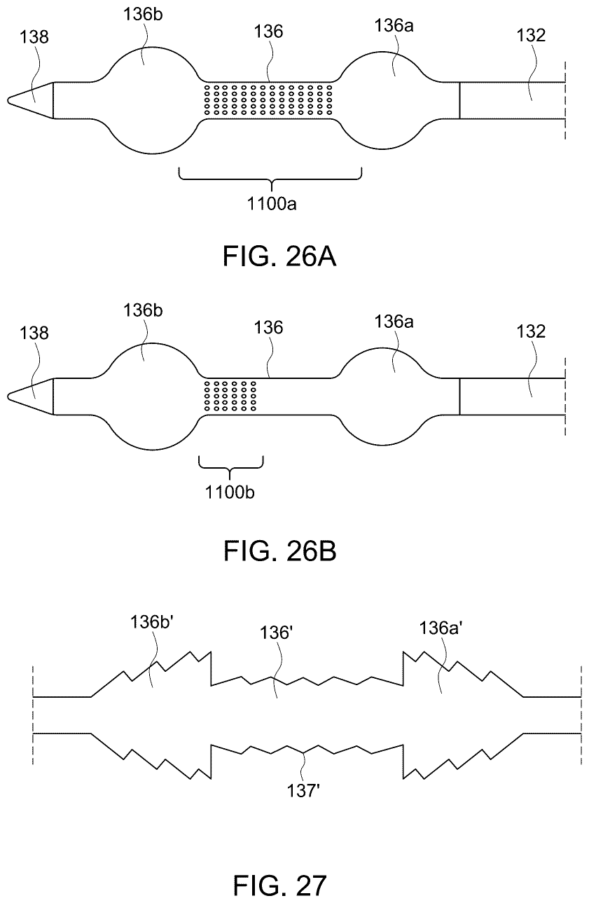

Now referring in addition to FIG. 26A, FIG. 26A shows an example of a distal end of delivery catheter 130 prior to the prosthetic heart valve 10 being crimped onto the balloon 136. Instead of including interior stops or valve retainers, the example of balloon 136 of FIG. 26A may include a high-friction surface 1100a to help maintain the axial position of the prosthetic heart valve 10 during delivery. In the illustrated example, as with other embodiments described herein, the balloon 136 may include a reduced diameter area between the proximal pillow 136a and the distal pillow 136b which is configured to receive the prosthetic heart valve 10 crimped thereon with opposite ends of the prosthetic heart valve abutting the proximal pillow 136a and the distal pillow 136b. In the illustrated example, in order to better help maintain the axial position of the prosthetic heart valve 10 on the balloon 136 during delivery, the high-friction surface 1100a may be positioned on the balloon 136 along substantially the entire length of the balloon 136 between the proximal pillow 136a and the distal pillow 136b. As used herein, the phrase “high-friction surface,” when used in relation to balloon 136, refers to the surface having a higher coefficient of friction compared to other areas of the balloon 136 that are not treated to have the high-friction surface. In one example, the high-friction surface 1100a may be created by coating the desired section of the balloon 136 with a higher friction material, such as coating the area of the balloon 136 between the proximal pillow 136a and the distal pillow 136b with polyurethane. As an alternative to or in addition to using a coating to increase the coefficient of friction, in another example, texturization (e.g., micro-texture including ridges, small bumps, cylindrical features, etc.) may be added to the desired section of the balloon 136. In some examples, this texturization may be created via 5-axis micro precision computer numerical control (“CNC”) milling, electrical discharge machining (“EDM”), lasers, electroforming, and/or other suitable techniques. Although the example of FIG. 26A includes the high-friction surface 1100a only along the entire length of the portion of the balloon 136 between the proximal pillow 136a and the distal pillow 136b, it should be understood that other options may be suitable. For example, in some alternatives, the entire length of the balloon 136 may be provided with the high-friction surface. In other examples, including the example shown in FIG. 26B, the high-friction surface 1100b may be provided on only a distal length of the balloon 136 between the proximal pillow 136a and the distal pillow 136b. For example, in some examples the high-friction surface 1100 may be provided on the distal half, distal third, distal quarter, etc. of the length of balloon 136 between the proximal pillow 136a and the distal pillow 136b. The example of FIG. 26B may provide inflow edge stability for the prosthetic heart valve 10 during delivery but also during deployment, which may be desirable. Although the examples described in connection with FIGS. 26A-B focus on exterior treatments to enhance axial stability of the prosthetic heart valve 10 relative to the balloon 136, it should be understood that any of the interior valve retainers or stops described herein may be combined with any of the exterior valve retainer embodiments described herein.

Now referring in addition to FIG. 27, whereas the examples described in connection with FIGS. 26A-26B may achieve high-friction surfaces via coatings, micro texturing, or the like, in other embodiments, including the example shown in FIG. 27, pleating may be used to create increased friction to assist with valve retention. For example, FIG. 27 shows an example of balloon 136′ that may be identical to balloon 136 (as well as have an identical use with delivery system 100), with one exception being that the balloon 136′ includes perpendicular pleats. In the illustrated example, balloon 136′ may include a proximal pillow 136a′ and a distal pillow 136b′ having similar or identical purposes and structures as proximal pillow 136a and distal pillow 136b. However, during manufacturing of the balloon 136′, pleats 137′ may be formed in balloon 136′ by creating folds (e.g., double or overlapping folds) with the pleats 137′ extending in a direction generally perpendicular or orthogonal to the central longitudinal axis of the balloon 136′. In other words, the pleats 137′ in some examples extend in a direction that encircles the central longitudinal axis of the balloon 136′. In some examples, the pleats 137′ may be formed prior to forming the balloon by blow-molding or after forming the balloon by blow-molding. In some examples, the pleats 137′ may be manually created and heat set so that, when the balloon 137′ is collapsed, the pleats 137′ are maintained. In some examples, the pleats 137′ are set so that, upon expansion of the balloon 137′, the pleats effectively disappear as the balloon 137′ reaches full expansion for deploying the prosthetic heart valve 10. In some examples, the pleats 137′ may serve as frictional ribs that increase the axial stability of the prosthetic heart valve 10 during delivery (e.g., reduce the likelihood of axial movement of the prosthetic heart valve 10 during delivery). In some examples, the pleats 137′ may be formed using a pillowing mold with a series of crevices for the pleats to form along with applying axial compression to push material into those crevices.

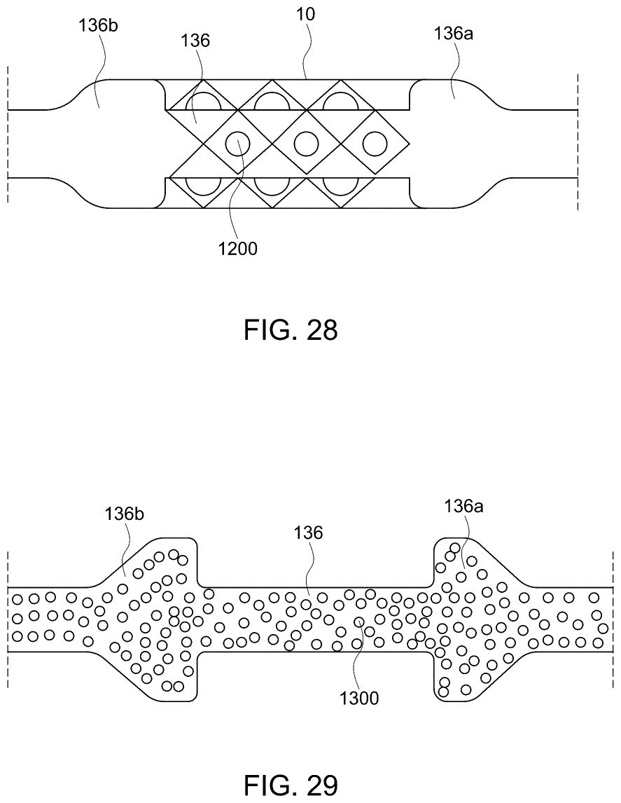

Now referring in addition to FIG. 28, FIG. 28 shows is an example of the distal end of balloon catheter 130 with the prosthetic heart valve 10 crimped thereon between the proximal pillow 136a and the distal pillow 136b. Rather than including a high-friction coating on the portion of the balloon 136 between the proximal pillow 136a and the distal pillow 136b, in the example of FIG. 28, a plurality of studs or nodules 1200 are provided on the central portion of the balloon 136 between the proximal pillow 136a and the distal pillow 136b. The studs or nodules 1200 are shown in FIG. 28 as being distributed in four columns oriented along the axial direction of the balloon 136, with each column being spaced about 90 degrees (+/−10 degrees) around the circumference of the balloon 136. However, it should be understood that the studs or nodules 1200 may be provided in other numbers and patterns. In some examples, the studs or nodules 1200 are formed separately from the balloon 136 and later fixed to the balloon 136. In other examples, the studs or nodules 1200 are formed integrally with the balloon 136, for example prior to or after forming the balloon 136 by blow-molding. In some examples, the studs or nodules 1200 may be molded individually and placed into nests surrounding the blow mold cavity prior to blowing the balloon. In such examples, the temperature of the chamber may chemically bond the studs or nodules 1200 onto the outer diameter of the balloon 136 when the balloon 136 is blown. When the prosthetic heart valve 10 is crimped over the balloon 136 between the proximal pillow 136a and the distal pillow 136b, the studs or nodules 1200 may contact the interior surface of the prosthetic heart valve 10, including for example the prosthetic leaflets 90, the inner skirt 60, and/or the frame 20, to increase friction on the prosthetic heart valve 10, making it more difficult for the prosthetic heart valve 10 to shift axially relative to the balloon 136 during delivery of the prosthetic heart valve 10. In some examples, the studs or nodules 1200 may be considered as macro-texturing, compared to the example of FIGS. 26A-26B that rely on micro-texturing.

Now referring in addition to FIG. 29, FIG. 29 is a highly schematic view of balloon 136 after having been filled with microbeads 1300. As shown in the example of FIG. 29, balloon 136 may be formed to have the same shape as many of the other balloons described herein, including a proximal pillow 136a and a distal pillow 136b. Prior to fixing the balloon 136 to other components of the delivery system 100 (such as to the nose cone 138 and the outer balloon catheter 132), the balloon 136 may be filled with a large quantity of microbeads 1300, which may be formed of silicone or another biocompatible material. In some examples, one end of the balloon 136 may be fixed to another component of the delivery device prior to filling the balloon 136 with microbeads 1300. In some examples, the microbeads 1300 may pack into the balloon 136 so that the balloon 136 becomes stiff prior to inflation. In these examples, the stiffness provided to the balloon 136 by the microbeads 1300 may further help prevent axial movement of the prosthetic heart valve 10 when the prosthetic heart valve 10 is mounted (e.g., crimped) on the balloon 136 with the axial ends of the prosthetic heart valve 10 in contact with the proximal pillow 136a and the distal pillow 136b. When the balloon 136 is inflated (e.g., via pressurized saline), the microbeads 1300 do not inhibit normal expansion of the balloon 136 and deployment of the prosthetic heart valve 10.

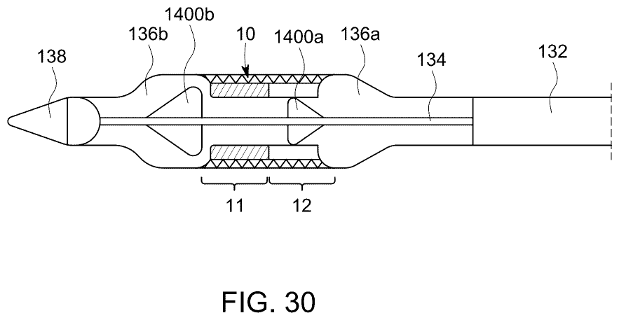

Now referring in addition to FIG. 30, FIG. 30 is a highly schematic cut-away view of a distal end of delivery catheter 130 in which prosthetic heart valve 10 has been crimped over balloon with axial ends of the prosthetic heart valve 10 in contact with proximal pillow 136a and distal pillow 136b. An expandable or non-expandable distal stop 1400b may be positioned within distal pillow 136b, which may in some examples be the same or similar (in both configuration and use) to distal stop 340. In some examples, distal stop 1400b may be omitted entirely. In the example of FIG. 30, a proximal stop 1400a is provided on the inner balloon catheter 134. However, unlike other examples of proximal stops described herein, proximal stop 1400a in some examples is not positioned within proximal pillow 136a, but is rather positioned between the proximal pillow 136a and the distal pillow 136b. In some examples, proximal stop 1400a is non-expandable, has a conical or frustoconical shape with the large-diameter end facing distally. In some examples, the largest outer diameter of the proximal stop 1400a is smaller than the inner diameter of proximal balloon leg 137a.

As shown in the example of FIG. 30, when prosthetic heart valve 10 is crimped over balloon 136, the mass of structure of the prosthetic heart valve 10 is not necessarily evenly distributed in the axial direction. In the example of FIG. 30, the outflow end of the prosthetic heart valve 10 may have less total bulk when considering the radial stack-up of frame 20, inner cuff 60, outer cuff 80, and prosthetic leaflets 90. For example, when the prosthetic heart valve 10 is crimped over balloon 136, a relatively low bulk zone 12 may be defined proximally while a relatively high bulk zone 11 may be defined distally. In some examples, the low bulk zone 12 may be positioned along the proximal half, proximal third, or proximal quarter of the length between the proximal pillow 136a and the distal pillow 136b. In some examples, the high bulk zone 11 may be positioned along the distal half, distal third, or distal quarter of the length between the proximal pillow 136a and the distal pillow 136b.

In the example of FIG. 30, the proximal stop 1400a is positioned along the inner balloon catheter 134 so that, when the prosthetic heart valve 10 is crimped over the balloon 136 between the proximal pillow 136a and the distal pillow 136b, the large-diameter distal end or distal face of the proximal stop 1400a is positioned at or proximally adjacent to the transition point between the low bulk zone 12 and the high bulk zone 11. With this example configuration, contact between the high bulk zone 11 of the prosthetic heart valve and the proximal stop 1400a (which contact may occur across the wall of the balloon 136) may provide axial stability against the prosthetic heart valve 10 sliding proximally relative to the balloon 136 during delivery of the prosthetic heart valve.

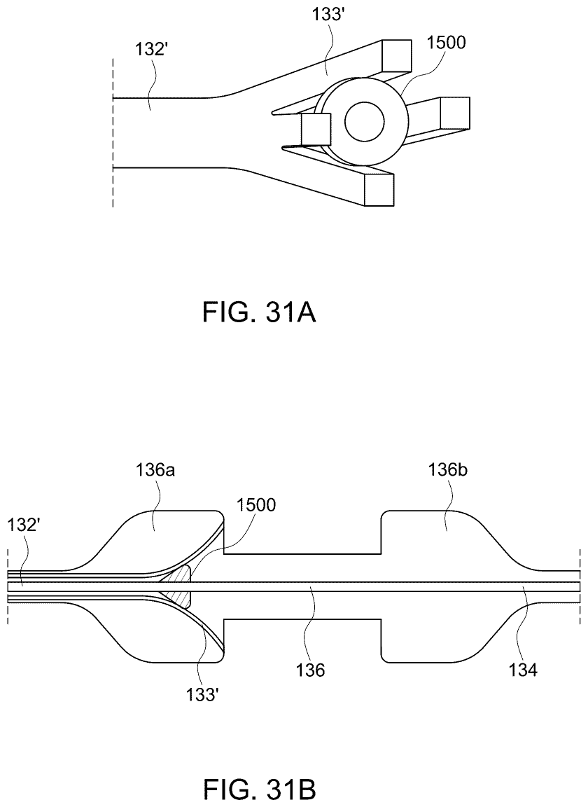

Now referring in addition to FIG. 31A, FIG. 31A is a highly schematic view of an example of a distal end of a spreadable outer balloon catheter 132′ being advanced over a spreader 1500. In some examples, outer balloon catheter 132′ may be similar or identical to outer balloon catheter 132, and may be used with delivery system 100 (or similar delivery systems) in place of outer balloon catheter 132, with at least one difference. For examples, whereas the distal end of outer balloon catheter 132 is a continuous cylinder like other portions of the catheter, the distal end of balloon catheter 132′ may include a plurality of axially-extending slits along the wall thickness of the balloon catheter 132′ that allow the distal end of the balloon catheter 132′ to have spreadable fingers 133′. In the example of FIG. 31A, the distal end of outer balloon catheter 132′ is being advanced distally over a ramped spreader feature 1500 causing the fingers 133′ to splay radially outwardly, as described in greater detail below in connection with FIG. 31B. It should be understood that, in some examples, it may be useful to make other modifications to outer balloon catheter 132′ to assist with allowing the fingers 133′ to splay. For example, outer balloon catheter 132 may include a plurality of layers when being formed, including at least one braided layer. In the example of FIGS. 31A-31B, it may be preferable to form the outer balloon catheter 132′ without any braided layers, or at least without a braided layer in the vicinity of the fingers 133′. As with other examples of outer balloon catheter 132 herein, outer balloon catheter 132′ may include one or more apertures similar to apertures 132a to assist with fluid flow.

Referring now in addition to FIG. 31B, FIG. 31B shows an example of the distal end of delivery catheter 130 that incorporates outer balloon catheter 132′ instead of outer balloon catheter 132. In the illustrated example, spreader 1500 may be coupled to inner balloon catheter 134 and be positioned within proximal pillow 136a. Spreader 1500 may be generally conical or frustoconical with the larger diameter end of the spreader 1500 positioned distally to the smaller diameter end of the spread 1500 to form a surface that is ramped in the distal direction. Prior to fixing the proximal leg 137a of balloon 136 to the balloon outer catheter 132′, the balloon outer catheter 132′ may be advanced through the proximal leg 137a. It should be understood that, in some examples, in the absences of applied forces that fingers 133′ form a generally cylindrical shape in combination that is similar to the shape of the remainder of the outer balloon catheter 132′. However, in the illustrated example, as the balloon outer catheter 132′ is advanced distally into the proximal pillow 136a and over spreader 1500, the ramped surface of the spreader 1500 forces the fingers 133′ to flare or splay radially outwardly. In some examples, the outer balloon catheter 132′ may be advanced distally relative to the inner balloon catheter 134 and the spreader 1500 until the fingers 133′ splay radially outwardly and are in contact with, or are proximally adjacent, the distal end of the proximal pillow 136a, for example similar to the configuration shown in FIG. 31B. During use, when the prosthetic heart valve 10 is crimped over the balloon 136 and positioned between the proximal pillow 136a and the distal pillow 136b, in some examples as the prosthetic heart valve 10 is delivered, the fingers 133′ provide axial support to the outflow end of the prosthetic heart valve 10, helping to ensure that the prosthetic heart valve 10 remains axially stable relative to the balloon 136 during delivery.

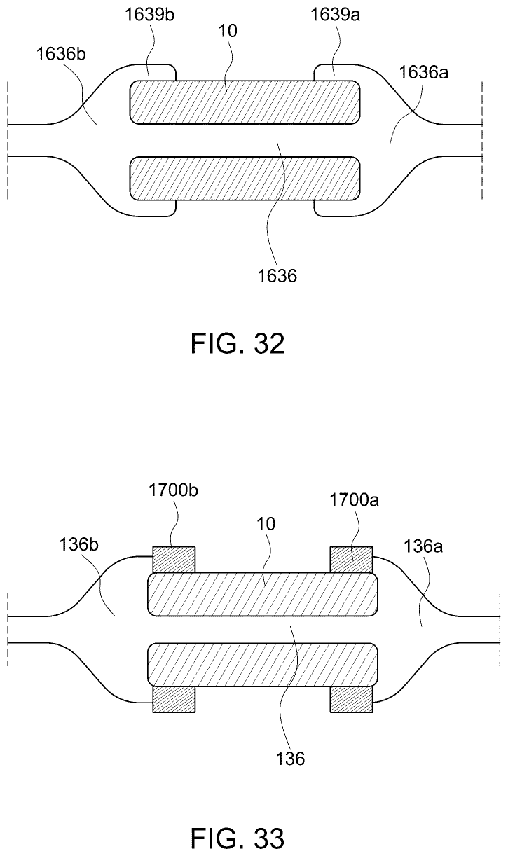

Now referring in addition to FIG. 32, FIG. 32 is a highly schematic cross-section of an example of a balloon 1636 that may be similar or identical to balloon 136, and used in place of balloon 136 in delivery system 100 (or similar delivery systems), with certain differences noted below. In some examples, the balloon 1636 includes a proximal pillow 1636a and a distal pillow 1636b that are similar in structure and purpose to counterpart proximal pillow 136a and distal pillow 136b. However, in some examples, whereas proximal pillow 136a and distal pillow 136b may only contact the crimped prosthetic heart valve 10 at its axial ends, proximal pillow 1636a and/or distal pillow 1363b may cover a portion of an outer or abluminal surface of the crimped prosthetic heart valve 10 for additional support. In the example of FIG. 32, proximal pillow 1636a includes a distally-extending overhang 1639a which may be generally cylindrical in shape. Similarly, in the example of FIG. 32, distal pillow 1636b includes a proximally-extending overhang 1639b which may be generally cylindrical in shape. It should be understood that in some embodiments, only one of the overhangs 1639a, 1639b may be included. If either overhang 1639a, 1639b is omitted from one of the pillows, the pillow may in some examples take the same or similar shape as its counterpart proximal pillow 136a or distal pillow 136b. In some examples the overhangs 1639a, 1639b may be formed into the corresponding pillow 1636a, 1636b during a process in which the pillow is being heat set. In other words, in some examples, the overhangs 1639a, 1639b may tend to take the shape shown in FIG. 32 in the absence of applied forces. In such examples, when crimping the prosthetic heart valve 10 over the balloon 1636, it may be helpful or necessary to temporarily pull the overhangs 1639a, 1639b in opposite directions to allow for clearance of the prosthetic heart valve 10 to be crimped onto the balloon 1636. In these examples, after the prosthetic heart valve 10 is crimped over the balloon 1636, the temporary force on the overhangs 1639a, 1639b may be removed so that they tend to move back to their set shape, including for example that shown in FIG. 32. In the illustrated example, in addition to providing axial stability to the prosthetic heart valve 10 via the axial ends of the prosthetic heart valve 10 contacting the corresponding pillows 1636a, 1636b, the overhangs 1639a, 1639b may provide additional protection via at least partial covering, and may also provide enhanced axial stability. Although various lengths of overhangs 1639a, 1639b may be suitable, in some examples, each overhang 1639a, 1639b that is incorporated into the balloon 1636 may extend an axial distance of between about 5% and about 15%, including about 10%, of the axial length of the crimped prosthetic heart valve 10. However, these numbers are merely exemplary, and lengths of less than 5% and greater than 0%, or above 15%, may in some examples be suitable. It should be understood that, in the example shown in FIG. 32 (and similarly configured examples), when the balloon 1636 is inflated to deploy the prosthetic heart valve 10, the overhangs 1639a, 1639b may effectively disappear as the balloon 1636 inflates, helping to ensure that the overhangs 1639a, 1639b do not interfere with deployment of the prosthetic heart valve 10.

Now referring in addition to FIG. 33, FIG. 33 is a highly schematic cross-section of an example of balloon 136 (e.g., as shown and described in connection with delivery system 100) that has been modified to include retainers 1700a, 1700b that may provide similar functionality to the overhangs 1639a, 1639b of FIG. 32. In some examples, the balloon 136 includes proximal pillow 136a and distal pillow 136b as described previously. In the example of FIG. 33, proximal pillow 136a includes a distally-extending retainer 1700a which may be generally cylindrical in shape. Similarly, in the example of FIG. 33, distal pillow 136b includes a proximally-extending retainer 1700b which may be generally cylindrical in shape. It should be understood that in some embodiments, only one of the retainers 1700a, 1700b may be included. Although retainers 1700a, 1700b may have a cylindrical shape, in some examples the retainers 1700a, 1700b may formed as a plurality of individual retainers 1700a, 1700b extending around the circumference of the corresponding pillow 136a, 136b. In some examples, if retainers 1700a, 1700b are provided as continuous cylindrical structures, they may inhibit the ability of the balloon 136 to expand. Thus, in some examples, each retainer 1700a, 1700b may be formed of two, three, four, or more individual retainers 1700a, 1700b that are not directly coupled to each other, with the plurality of individual retainers 1700a, 1700b spaced from each other around the circumference of the corresponding pillow 136a, 136b. In some examples, the retainers 1700a, 1700b may be formed of a rigid material, such as a polymer, that can be fixed to the corresponding pillow 136a, 136b by adhesives, overmolding, or any other suitable method. In some examples, the retainers 1700a, 1700b may be formed of an amide polymer such as Pebax or Nylon, which may be bonded to the balloon 136 when overmolded. The retainers 1700a, 1700b may in some examples function similarly to the overhangs 1639a, 1639b described above. For example, when crimping the prosthetic heart valve 10 over the balloon 136, it may be helpful or necessary to temporarily move the retainers 1700a, 1700b in opposite directions to allow for clearance of the prosthetic heart valve 10 to be crimped onto the balloon 136. In the illustrated example, in addition to providing axial stability to the prosthetic heart valve 10 via the axial ends of the prosthetic heart valve 10 contacting the corresponding pillows 136a, 136b, the retainers 1700a, 1700b may provide additional protection via at least partial covering, and may also provide enhanced axial stability. Although various lengths of retainers 1700a, 1700b may be suitable, in some examples, each retainer 1700a, 1700b that is incorporated into the balloon 136 may extend an axial distance of between about 5% and about 15%, including about 10%, of the axial length of the crimped prosthetic heart valve 10. However, these numbers are merely exemplary, and lengths of less than 5% and greater than 0%, or above 15%, may in some examples be suitable. It should be understood that, in the example shown in FIG. 33 (and similarly configured examples), when the balloon 136 is inflated to deploy the prosthetic heart valve 10, the prosthetic heart valve 10 will tend to axially foreshorten as it expands radially, and the axial foreshortening may help ensure that the retainers 1700a, 1700b do not interfere with deployment of the prosthetic heart valve 10.

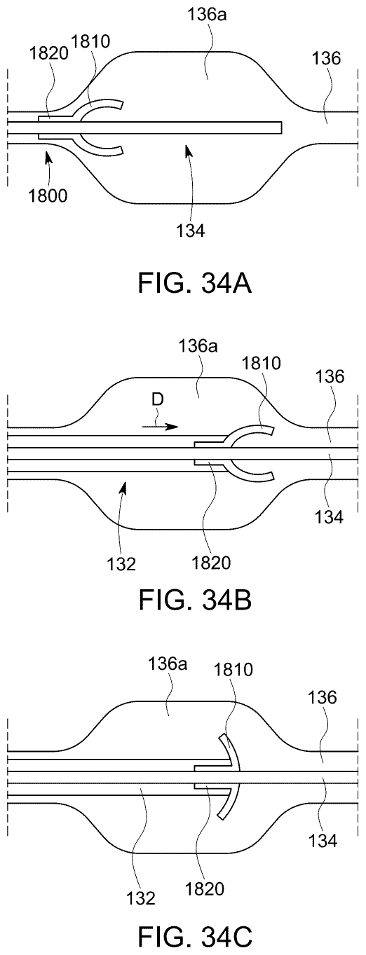

Now referring in addition to FIGS. 34A-34C, FIGS. 34A-34C are highly schematic cross-sections of example methods of forming an example of a stop 1800 within a balloon 136 that may include or more of the following steps. In the example view of FIG. 34A, the proximal pillow 136a is shown with the inner balloon catheter 134 being inserted through the interior of the balloon 136, in a distal direction, during assembly. The stop 1800 in this example is fixed to the inner balloon catheter 134, and may include a distal retaining surface 1810 and a proximal shaft 1820. In some examples, the proximal shaft 1820 is fixed to the inner balloon catheter 134, for example via adhesives, while the retaining surface 1810 is not directly fixed to the inner balloon catheter 134. During an initial state of assembly, including the example shown in FIG. 34A, the retaining surface 1810 may be temporarily configured to have a cup-shape or leading concave surface in which the retaining surface 1810 has a generally “C”- or “U”-shaped profile, with the open end of the “C”- or “U”-shaped profile facing distally. In the illustrated example, when the retaining surface 1810 of stop 1800 has this temporary cup-shape, the maximum outer diameter of the stop 1800 may be smaller than the inner diameter of the proximal balloon leg 137a, allowing the stop 1800 to readily pass through the proximal balloon leg 137a and into the interior of the balloon 136 during manufacturing.

Now referring in addition to FIG. 34B, FIG. 34B illustrates an example of a second stage of assembly occurring after that shown in FIG. 34A, in which the inner balloon catheter 134 has been advanced in the distal direction D to its final desired position, which may correspond to the configuration in which the stop 1800 is positioned at or adjacent to the distal end of the proximal pillow 136a. In the example configuration of FIG. 34B, the retaining surface 1810 of stop 1800 is still in the temporary distally-facing cup shape, and the outer balloon catheter 132 has been advanced in the distal direction D until it is overlying the proximal shaft 1820 of the stop 1800 but just prior to contacting the retaining surface 1810.

Now referring in addition to FIG. 34C, FIG. 34C illustrates an example of a third stage of assembly occurring after that shown in FIG. 34B. In particular, in this example, between the stages shown in FIG. 34B and FIG. 34C, the outer balloon catheter 132 was slightly advanced in the distal direction D until the distalmost end of the outer balloon catheter 132 applied pressure to the proximal surface of the retainer surface 1810, causing the retainer surface 1810 to revert to a mushroom cap shape om which the leading or distal end of the retainer surface 1810 has a slight convexity. In some examples, the entire stop 1800 or at least the retainer surface 1810 is formed of a semi-compliant material (for example such as a Pebax material or a thermoplastic polyurethane “TPU”) that is set (e.g., via heat treatment) to have the mushroom-shape shown in FIG. 34C in the absence of applied forces (i.e. “mushroom shape” including a rectangular stem portion interconnected to a domed, cup shape portion that has a width that is greater than a width of both the outer balloon catheter 132 and the stem portion). It should be understood that, in some examples, although the retainer surface 1810 is set to have the mushroom shape of FIG. 34C in the absence of applied forces, it may be effectively inverted manually prior to the first assembly step shown in the example of FIG. 34A, and the inverted cup shape may be stable enough to retainer that cup shape until additional forces are applied. In this example, as the outer balloon catheter 132 is driven distally into the proximal side of the retainer surface 1810, similar to the configuration shown in FIG. 34B, the applied forces from the outer balloon catheter 132 disrupts the semi-stable cup shape shown in FIG. 34B, allowing the retainer surface 1810 to “snap” back or revert to the set mushroom shape shown in FIG. 34C. In some examples, when the retainer surface 1810 has the mushroom shape of FIG. 34C, it has a maximum outer diameter that is larger than the inner diameter of the proximal balloon leg 137a, allowing the stop 1800 to have a relatively large outer diameter to provide axial stability to a prosthetic heart valve 10 mounted to the balloon 136 in axial contact with the proximal pillow 136a.

Although many examples of valve retainers and/or stops are described herein, they my all provide a substantially similar functionality in the overall process of delivering and deploying a prosthetic heart valve such as prosthetic heart valve 10. For example, referring briefly to the example method 200 of FIG. 11, during some examples of step 206 of advancing the delivery catheter (e.g., delivery catheter 130) over a guidewire, the delivery catheter is advanced through an introducer with a hemostasis valve. In such examples, any of the valve retainers or stops described herein (including examples in which a high-friction feature is provided on the balloon (e.g., the examples of FIGS. 26A-28) and examples in which the outer balloon catheter itself provides valve-retaining functionality (e.g., the example of FIG. 17)) may help prevent the prosthetic heart valve from axial movement relative to the balloon (e.g., balloon 136), including helping to prevent proximal movement as the delivery catheter is advanced distally. This example functionality may also be provided at other stages of delivery, including for example when tracking around the aortic arch in step 208 if the procedure is a transfemoral aortic valve replacement. Further, as noted above, although protection against proximal movement of the prosthetic heart valve during distal advancement may in some examples be more important than protection against distal movement of the prosthetic heart valve, the inclusion of any of the distal stops (including high-friction surfaces described here) may be helpful in preventing such axial movement. Still further, it should be understood that different classes of examples may be combined. For example, features are described herein that involve (i) modification of the outer surface of a balloon (e.g., high-friction examples of FIGS. 26A-28), (ii) placing physical stops or valve retainers within a proximal pillow, (iii) placing physical stops or valve retainers within a distal pillow, (iv) placing physical stops or valve retainers within a crimp zone between proximal and distal pillows (e.g., the example of FIG. 30), and (v) adding features to the balloon that will at least partially cover an outer surface of the prosthetic heart valve (e.g., the examples of FIGS. 32-33) may be combined. It should be understood that any of the examples of classes (i), (ii), (iii), (iv), and (v) may be combined with any examples of the other classes in a single device. As one illustrative example, the overhangs 1639a, 1639b of FIG. 32 may be used in a system having a high-friction surface 1100a of FIG. 26A, along with a stop 800 of FIGS. 23A-23B used within a distal pillow 136b and a proximal stop 400a of FIGS. 18A-18B used within a proximal pillow 136a in addition to the stop 1400a of FIG. 30 positioned inside of the crimped prosthetic heart valve 10. This is just one example to show how different classes of retaining features described herein may be combined into a single device, and other suitable combinations would be readily apparent to the skilled artisan.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for delivering a prosthetic heart valve, the system comprising:
a handle;
an outer catheter extending from the handle;
an inner catheter extending from the handle and through an interior of the outer catheter;
a nose cone coupled to a distal end of the inner catheter;
an inflatable balloon having a proximal leg coupled to the outer catheter, and a distal leg coupled to (i) the nose cone or (ii) the inner catheter; and
a first valve retainer coupled to the inner catheter and positioned within the balloon, the first valve retainer having an assembled state in which an inner stop is directly fixed to the inner catheter and an outer stop is positioned around and coupled to the inner stop,
wherein the inner stop has a maximum outer diameter that is smaller than a maximum inner diameter of the distal leg, and in the assembled condition of the first valve retainer, the outer stop has a maximum outer diameter that is larger than the maximum inner diameter of the distal leg.

2. The system of claim 1, wherein the inner stop includes a ramped outer surface portion that increases in diameter in a proximal-to-distal direction of the inner stop.

3. The system of claim 2, wherein the outer stop includes a rim and a plurality of extensions extending from the rim.

4. The system of claim 3, wherein the outer catheter is directly coupled to the rim.

5. The system of claim 3, wherein when the first valve retainer is in an unassembled condition, and in the absence of applied forces, the outer stop has a maximum outer diameter that is smaller than the maximum inner diameter of the distal leg.

6. The system of claim 5, wherein the inner stop includes a shoulder at a distal end of the ramped outer surface portion.

7. The system of claim 6, wherein at least one of the plurality of extensions includes an interiorly extending prong at a distal end thereof, the interiorly extending prong configured to engage the shoulder in the assembled condition of the first valve retainer.

8. The system of claim 3, wherein in the assembled condition of the first valve retainer, each of the plurality of extensions flares radially outwardly relative to the rim.

9. The system of claim 8, wherein each of the plurality of extensions is coupled to the rim via a living hinge.

10. The system of claim 1, wherein the balloon includes a proximal pillow, a distal pillow, and a central portion between the proximal pillow and the distal pillow, the proximal pillow and the distal pillow each having a diameter that is greater than a diameter of the central portion, the central portion being configured to receive the prosthetic heart valve in a crimped condition.

11. The system of claim 10, wherein the first valve retainer is positioned inside the proximal pillow.

12. The system of claim 10, wherein a distal end of the outer catheter extends into the proximal pillow.

13. The system of claim 12, wherein the outer catheter includes a catheter wall, and a plurality of apertures are formed within the catheter wall, the plurality of apertures being positioned within the proximal pillow.

14. A method of manufacturing a delivery catheter, the method comprising:
providing a balloon having a distal leg and a proximal leg;
inserting an inner catheter into an interior of the balloon so that an inner stop fixed to the inner catheter passes through the distal leg, the inner stop having a maximum outer diameter that is smaller than a maximum inner diameter of the distal leg;
inserting an outer stop into the interior of the balloon so that the outer stop passes through the distal leg while the inner catheter is received through the outer stop, wherein during inserting the outer stop, the outer stop has a maximum outer diameter that is smaller than the maximum inner diameter of the distal leg; and
while the inner stop and the outer stop are both within the interior of the balloon, advancing the outer stop over the inner stop to increase the maximum outer diameter of the outer stop, the increased maximum outer diameter of the stop being larger than the maximum inner diameter of the distal leg.

15. The method of claim 14, wherein the inner stop has a ramped outer surface portion and the outer stop has a rim and a plurality of extensions extending from the rim, and advancing the outer stop over the inner stop includes advancing the outer stop over the ramped outer surface portion to force the plurality of extensions to flare outwardly relative to the rim.

16. The method of claim 15, wherein the inner stop includes a shoulder, and at least one of the plurality of extensions includes an interiorly extending prong.

17. The method of claim 16, wherein in the absence of applied forces, the outer stop has the maximum outer diameter that is smaller than the maximum inner diameter of the distal leg.

18. The method of claim 17, further comprising continuing to advance the outer stop over the inner stop until the at least one interiorly extending prong advances past the shoulder.

19. The method of claim 18, wherein when the at least one interiorly extending prong advances past the shoulder, the corresponding extension snaps inwardly toward the inner catheter such that the at least one interiorly extending prong engages the shoulder to prevent the outer stop from retracting relative to the inner stop.

20. The method of claim 19, further comprising advancing an outer catheter through the proximal leg into the interior of the balloon.
